(19) United States
(12) Patent Application Publication
(10) Pub. No.: US 2026/0210942 A1
(43) Pub. Date: Jul. 23, 2026

(54) METHOD AND DEVICE FOR DETECTING A CELL RESPONSE

(71) Applicant: 100XBIO, INC., Woburn, MA (US)

(72) Inventor: Sergei PUSTYLNIKOV, Stoneham, MA (US)

(21) Appl. No.: 19/444,363

(22) Filed: Jan. 9, 2026

Related U.S. Application Data
(63) Continuation of application No. PCT/US24/37718, filed on Jul. 12, 2024.
(60) Provisional application No. 63/526,370, filed on Jul. 12, 2023.

(51) Int. Cl. G01N 33/50; G01N 33/58; G01N 35/00

(52) U.S. Cl. CPC G01N 33/505; G01N 33/5014; G01N 33/582; G01N 35/00029; G01N 2035/00099

(57) ABSTRACT

Described are systems and methods for identifying a cellular response to a candidate agent comprising treatment cycling. The systems and method utilize quick response readouts in combination with gentle removal and/or exchange of the analyte fluid. The systems and methods can, for example, be used for T-cell epitope mapping, T-cell diagnostics and other cell-based high-throughput screening methods.

### RELATED APPLICATIONS

This application is a continuation of International Application No. PCT/US2024/037718, which designated the United States and was filed on Jul. 12, 2024, published in English, which claims the benefit of U.S. Provisional Application No. 63/526,370 filed Jul. 12, 2023. The entire teachings of the above-referenced applications are incorporated herein by reference.

### BACKGROUND OF THE INVENTION

In ex vivo and in vitro biological testing, small specimen volume and underrepresentation of rare cell types represent substantial limitations, especially when working with the primary cells derived from patients or experimental animals and in screening up to and over 1000 treatment conditions (drug screening, receptor specificity studies). An example of such a limitation is in the screening of precise peptide specificity of the T cell receptors (TCRs).

T-cell activation is critical for the initiation of the immune response and includes three stages. The first stage involves processing and presentation of antigens as epitopes on the surface of the antigen-presenting cells (APC) in a complex with major histocompatibility complex (MHC) molecules. The second stage involves binding between an antigen-specific T cell receptor (TCR) and the antigen-MHC complex. This leads to the formation of the cell-to-cell interaction between the T cell and APC called immune synapse. The third stage involves intracellular signaling and the “per se” activation of the T cells, leading to their change in phenotype, secretion of the cytokines, migration and proliferation. ELISpot (enzyme-linked immunospot) and flow cytometry (Fluorescence-activated Cell Sorting, FACS) combined with intracellular cytokine staining (ICS) or staining for activation-induced markers (AIM) are common research methods used to detect T-cell activation. ELISpot usually detects cytokine spots generated around the cytokine-secreting cells using a specific capture antibody and a detection antibody. Flow cytometry detects either cytokine-secreting T cells or the T cells that start to express specific activation-induced markers on their surface.

An antigen recognized by a given T cell is typically present at exceedingly low frequencies, e.g., 1 in 100,000 antigens or less. In addition, not every APC displaying a given antigen will encounter a T cell with specificity for that specific APC. As such, the probability of a positive response is low. For example, using traditional research methods, the probability of a positive response is 0.01% or lower in the individual experimental condition, with over 99.99% of the specimen used to obtain the negative readouts. Thus, traditional research methods are associated with practical limitations including complicated logistics and high costs.

There remains a need in the art for high-throughput methods and devices for identifying such low-probability positive responses using a small volume of biological sample.

### SUMMARY OF THE INVENTION

The present invention is at least partially based on the discovery of a method allowing multiple quick cycles testing different candidate agents against the same small volume of sample (for example, wherein the sample comprises cells). The methods described herein can be used for T-cell epitope mapping, T-cell diagnostics and other cell-based high-throughput screening methods. The described systems and methods employ quick response readouts (e.g., less than 1 hour) in combination with gentle removal, optional washing of the analyte fluid, and treatment exchange, and. The systems and methods combine desired features of fluorescence microscopy and/or label-free or labeled-based readouts with gentle removal/wash or treatment exchange methods to provide high throughput, and/or automated methods and systems for screening candidate agents against a small volume of sample comprising cells. The systems and methods described herein can also be used to identify cells and/or the number cells that respond to an analyte, such as a library of analytes.

The invention encompasses a method of detecting a cellular response to a candidate agent, the method comprising:
i. a first cycle comprising:
   a. contacting a sample comprising cells with an analyte fluid, wherein the sample comprising the cells is on a solid support and wherein the analyte fluid comprises a candidate agent in a liquid medium;
   b. detecting the cellular response by detection of an optical signal in the sample on the solid support; and
   c. removing the analyte fluid from the solid support thereby removing the candidate agent while retaining the sample comprising the cells on the solid support;
ii. a subsequent cycle comprising:
   a. contacting the retained sample comprising the cells of the preceding cycle with a new analyte fluid, wherein the new analyte fluid comprises a new candidate agent in a liquid medium, wherein the new candidate agent is different from the candidate agent of the preceding cycle;
   b. detecting the cellular response by detection of an optical signal in the retained sample on the solid support; and
   c. removing the analyte fluid from the solid support thereby removing the candidate agent while retaining the sample comprising the cells on the solid support; repeating the subsequent cycle at least N times, wherein N is an integer greater than or equal to 0, wherein each new analyte fluid of each subsequent cycle comprises a different candidate agent from that of the preceding cycle. The removal step is performed using a gentle removal/wash method and/or treatment exchange as described herein. In certain aspects, the removal of the analyte fluid (e.g., by treatment exchange or gentle wash) is a method whereby the cell viability and/or the cell quantity of the retained sample is decreased by no more than about 5% after 2 to 10, or 10 or more, total cycles and that does not comprise high-speed centrifugation. For example, the cell viability and/or the cell quantity of the retained sample is decreased by no more than about 5% after 2, 3, 4, 5, 6, 7, 8, 9, 10 or more total cycles. In some embodiments, the cells are non-adherent cells and the cell viability and/or the cell quantity of the retained sample is decreased by no more than about 5% after 2, 3, 4, 5, 6, 7, 8, 9, 10 or more total cycles. As used herein, high-speed centrifugation is centrifugation at a centrifugal force over 250×g. In certain additional aspects, the removal of the analyte fluid is a method whereby the cell viability and/or the cell quantity of the retained sample is decreased by no more than about 5% after 10 total cycles and that does not comprise centrifugation. Non-limiting examples of cells that can be used in the method are immune cells. Immune cells include, for example, B cells, T cells, innate lymphoid cells, natural killer cells, natural killer T cells, gamma delta T cells, macrophages, monocytes, dendritic cells, neutrophils, myeloid derived suppressor cells, hematopoietic stem cells, or mesenchymal stem cells, induced pluripotent stem cells (iPSCs) and iPSC-derived cells. In some embodiments, the population of immune cells is one or more of B cells, T cells, innate lymphoid cells, natural killer cells, natural killer T cells, gamma delta T cells, T regulatory cells, macrophages, monocytes, dendritic cells, neutrophils, myeloid derived suppressor cells. Yet additional examples of cells that can be used in the method are mixed immune and non-immune cell cultures and suspensions, such as blood cells, peripheral blood mononuclear cells, lymph node cells, mucus samples, tumor cell suspensions and tumor-infiltrating leukocytes (TILs). In certain aspects, the sample is multiple samples and optionally, the multiple samples are on a multi-well plate. For example, the multiple samples (comprising the cells) are on the membranes of transwell inserts and each insert is placed in a well of the multi-well plate.

The invention also encompasses a method of identifying a candidate agent, the method comprising:
iii. a first cycle comprising:
   a. contacting a sample comprising cells with an analyte fluid, wherein the sample comprising the cells is on a solid support and wherein the analyte fluid comprises a candidate agent in a liquid medium;
   b. detecting the response by detection of an optical signal in the sample on the solid support; and
   c. removing the analyte fluid from the solid support thereby removing the candidate agent while retaining the sample comprising the cells on the solid support;
iv. a subsequent cycle comprising:
   a. contacting the retained sample comprising the cells of the preceding cycle with a new analyte fluid, wherein the new analyte fluid comprises a new candidate agent in a liquid medium, wherein the new candidate agent is different from the candidate agent of the preceding cycle;
   b. detecting the response by detection of an optical signal in the retained sample on the solid support; and
   c. removing the analyte fluid from the solid support thereby removing the candidate agent while retaining the sample comprising the cells on the solid support;

repeating the subsequent cycle at least N times, wherein N is an integer greater than or equal to 0, wherein each new analyte fluid of each subsequent cycle comprises a different candidate agent from that of the preceding cycle. The removal step is performed using a gentle removal/wash method as described herein. In certain aspects, the removal of the analyte fluid is a method whereby the cell viability and/or the cell quantity of the retained sample is decreased by no more than about 5% after 10 total cycles and that does not comprise high-speed centrifugation. As used herein, high-speed centrifugation is centrifugation at a centrifugal force over 250×g. In certain additional aspects, the removal of the analyte fluid is a method whereby the cell viability and/or the cell quantity of the retained sample is decreased by no more than about 5% after 10 total cycles and that does not comprise centrifugation. Non-limiting examples of cells that can be used in the method are immune cells. Immune cells include, for example, B cells, T cells, innate lymphoid cells, natural killer cells, natural killer T cells, gamma delta T cells, macrophages, monocytes, dendritic cells, neutrophils, myeloid derived suppressor cells, hematopoietic stem cells, or mesenchymal stem cells, induced pluripotent stem cells (iPSCs) and iPSC-derived cells. In some embodiments, the population of immune cells is one or more of B cells, T cells, innate lymphoid cells, natural killer cells, natural killer T cells, gamma delta T cells, T regulatory cells, macrophages, monocytes, dendritic cells, neutrophils, myeloid derived suppressor cells. Yet additional examples of cells that can be used in the method are mixed immune and non-immune cell cultures and suspensions, such as blood cells, peripheral blood mononuclear cells, lymph node cells, mucus samples, tumor cell suspensions and tumor-infiltrating leukocytes (TILs).

In certain aspects of the methods described herein, the cellular response is detected less than 6 hours, less than 4 hours, less than 2 hours, less than 1 hour or less than 30 minutes, less than 15 minutes or less than 15 minutes after the cells are contacted with the analyte fluid.

In additional aspects, the method is a method of detecting T-cell activation, the method comprising:
i. a first cycle comprising:
   a. contacting a sample comprising T-cells with an analyte fluid, wherein the sample comprising the T-cells is on a solid support and wherein the analyte fluid comprises a candidate agent in a liquid medium;
   b. detecting the T-cell activation by detection of an optical signal in the sample on the solid support; and
   c. removing the analyte fluid from the solid support thereby removing the candidate agent while retaining the sample comprising the T-cells on the solid support;
ii. a subsequent cycle comprising:
   a. contacting the retained sample comprising the T-cells of the preceding cycle with a new analyte fluid, wherein the new analyte fluid comprises a new candidate agent in a liquid medium, wherein the new candidate agent is different from the candidate agent of the preceding cycle;
   b. detecting the T-cell activation by detection of an optical signal in the retained sample on the solid support; and
   c. removing the analyte fluid from the solid support thereby removing the candidate agent while retaining the sample comprising the T-cells on the solid support; repeating the subsequent cycle at least N times, wherein N is an integer greater than or equal to 0, wherein each new analyte fluid of each subsequent cycle comprises a different candidate agent from that of the preceding cycle. The removal step is performed using a gentle wash and/or treatment exchange method as described herein. In certain aspects, the removal of the analyte fluid is a method whereby the cell viability and/or the cell quantity of the retained sample is decreased by no more than about 5% after 10 total cycles and that does not comprise high-speed centrifugation. In certain additional embodiments, the removal of the analyte fluid is a method whereby the cell viability and/or the cell quantity of the retained sample is decreased by no more than about 5% after 10 total cycles and that does not comprise centrifugation. In certain aspects, the sample is multiple samples and optionally, the multiple samples are on a multi-well plate. For example, the multiple samples (comprising the cells) are on the membranes of transwell inserts and each insert is placed in a well of the multi-well plate.

In additional aspects, the method is a method of identifying a candidate agent that activates T-cells, the method comprising:
i. a first cycle comprising:
   a. contacting a sample comprising T-cells with an analyte fluid, wherein the sample comprising the T-cells is on a solid support and wherein the analyte fluid comprises a candidate agent in a liquid medium;
   b. detecting T-cell activation by detection of an optical signal in the sample on the solid support; and
   c. removing the analyte fluid from the solid support thereby removing the candidate agent while retaining the sample comprising the T-cells on the solid support;
ii. a subsequent cycle comprising:
   a. contacting the retained sample comprising the T-cells of the preceding cycle with a new analyte fluid, wherein the new analyte fluid comprises a new candidate agent in a liquid medium, wherein the new candidate agent is different from the candidate agent of the preceding cycle;
   b. detecting T-cell activation by detection of an optical signal in the retained sample on the solid support; and
   c. removing the analyte fluid from the solid support thereby removing the candidate agent while retaining the sample comprising the T-cells on the solid support;

repeating the subsequent cycle at least N times, wherein N is an integer greater than or equal to 0, wherein each new analyte fluid of each subsequent cycle comprises a different candidate agent from that of the preceding cycle. The removal step is performed using a gentle wash/treatment exchange method as described herein. In certain aspects, the removal of the analyte fluid is a method whereby the cell viability and/or the cell quantity of the retained sample is decreased by no more than about 5% after 10 total cycles and that does not comprise high-speed centrifugation. In certain additional embodiments, the removal of the analyte fluid is a method whereby the cell viability and/or the cell quantity of the retained sample is decreased by no more than about 5% after 10 total cycles and that does not comprise centrifugation.

The methods described herein allows detection of multiple events of low probability within the same sample. For example, the method allows detection of different subpopulations of cells (such as T cells) responding specifically to different treatments (such as treatments comprising their cognate peptides). The methods can be either more sample-efficient and/or more sensitive, e.g., for users that prefer not to decrease sample volume. For some users, the methods described herein can be simultaneously more sample-efficient and more sensitive.

In some embodiments, the cells are incubated for a time sufficient for the cells to respond (e.g., a time sufficient to activate the T-cells). In certain aspects, the response (a positive readout or negative readout) is detected in less than about 6 hours, less than about 4 hours, less than about 2 hours, less than about 1 hour or less than about 30 minutes, less than about 15 minutes, less than 10 minutes, or less than about 5 minutes after the cells are contacted with the analyte fluid. The positive readout and/or negative readout for a sample during a specific cycle can be recorded. In certain aspects, a cycle includes more than response detection steps, e.g., detection of the optical signal at different time points and/or detection of different optical signals.

In certain aspects, a sample for which a response (e.g., T-cell activation) is detected is further characterized using a downstream assay. In certain aspects, the downstream assay is conducted or carried out on the same support as the treatment cycling. In yet additional aspects, the staining and/or imaging of the downstream assay is performed by the same device that carries out the treatment exchange. In some examples, the downstream assay is automated. One specific method recommended as a downstream assay is a post-cycling automated staining of live or fixed cells with dyes or antibodies (e.g., a labeled antibody), that confirms or enriches cycling data, is a method referred herein as “AIMSpot.” In certain embodiments, the downstream assay is an activation-induced marker (AIM) assay.

AIMSpot is a method that combines treatment cycling with automated or semi-automated immunofluorescent staining or other imaging techniques to detect an activation-induced marker. AIMSpot is a research method that comprises (a) a cell culture system where imaging and treatment exchange for live cell culture allows tracking individual cells without cell loss and without significant changes of cellular locations, (b) live cell culture observation system with detection of individual cellular features and responses over time, and (c) a system or method for downstream (post-cycling) staining and characterization of additional cellular features and markers in the same traceable cells or at their corresponding locations; post-cycling characterization can be done with live or fixed cells. The cellular features can include phenotypic markers (such as CD4, CD8 and others), expressed cytokines or other secretory molecules captured within the cells (such as IFN-g, IL-2, TNF and others; perforin, granzyme B and others), expressed activation-induced markers (collectively called AIM, such as CD69, 4-1BB, OX40, CD40L and others).

It should be understood that a method wherein “the removal of the analyte fluid is a method whereby the cell viability and/or the cell quantity of the retained sample is decreased by no more than about 5% after 10 total cycles” or “the removal of the analyte fluid is a method whereby the cell viability and/or the cell quantity of the retained sample is would be decreased by no more than about 5% after 10 total cycles” does not mean that the method requires at least 10 total cycles (or that N is at least 8), but rather, that the method of removing the analyte fluid is one that would result in a decrease of no more than about 5% if 10 total cycles were completed. Thus, in one example, N is 1 (e. g, there are 3 total cycles) and the removal of the analyte fluid is a method whereby the cell viability and/or the cell quantity of the retained sample would be decreased by no more than about 5% if 10 total cycles were completed and that does not comprise centrifugation. In another example, N is 15 and the removal of the analyte fluid is a method whereby the cell viability and/or the cell quantity of the retained sample is decreased by no more than about 5% if 10 total cycles were completed and that does not comprise centrifugation.

In certain aspects, N (the number of cycles in addition to the first and second cycle) is about 0 or more, 1 or more, 2 or more, about 5 or more, about 10 or more, about 15 or more, about 20 or more, about 30 or more, about 40 or more, about 49 or more, about 50 or more, about 74 or more, about 75 or more, about 99 or more, about 100 or more, or about 200 or more. In further aspects, the total number of cycles (N+2) is about is about 2 or more, 3 or more, about 5 or more, about 10 or more, about 15 or more, about 20 or more, about 30 or more, about 40 or more, about 49 or more, about 50 or more, about 75 or more, about 100 or more, or about 200 or more. The number of cycles is tailored for the specific sample and the specific response read out. For example, in an automated system, the number of cycles can be programmed depending on the specific sample and the specific response read-out being detected.

The methods described herein can be automated and/or high-throughput.

The invention additionally encompasses an automated system or device for performing a method described herein.

In one embodiment, the invention is an automated system or device for identifying a candidate agent that elicits a response in a cell, wherein the system comprises:
a. a dispensing unit configured for dispensing the analyte fluid onto the solid support; for example, samples comprising cells are in the wells of a multi-well plate or on transwell inserts placed in the wells of a multi-well plate and the dispensing unit is configured for dispensing an analyte fluid into each well of the multi-well plate;
b. an optical imaging unit configured for detecting the cell response by detection of an optical signal; and
c. a washing unit configured for removing the analyte fluid from the solid support (e.g., each well) thereby removing the candidate agent and retaining the sample comprising the cells on the solid support.

In certain specific aspects, the automated system or device comprises a multi-well plate. The multi-well plate can comprise a transwell insert. In another example, the multi-well plate does not include a transwell insert. The multi-well plate is configured such that the cells can be exposed to more than one treatment cycle and such that the location of individual cells does not change over the different treatment cycles. It should be understood that each “unit” can be distinct or combined with another unit, for example, the washing unit can be part of the dispensing unit, or in other words the same element of the device dispenses the analyte and removes the analyte. In additional aspects, the multi-well plate is configured such that the cells can be exposed to more than one treatment cycle and such that the relative location of individual cells does not significantly change over the different treatment cycles.

In certain aspects, the invention is an automated system or device for identifying T-cell activation, wherein the system comprises:
a. a dispensing unit configured for dispensing the analyte fluid onto a solid support; for example, samples comprising T-cells are in the wells of a multi-well plate or on transwell inserts placed in the wells of a multi-well plate and the dispensing unit is configured for dispensing an analyte fluid into each well of the multi-well plate;
b. an optical imaging unit configured for detecting the T-cell activation response by detection of an optical signal; and
c. a washing unit configured for removing the analyte fluid from the solid support, e.g., each well thereby removing the candidate agent and retaining the sample comprising the T cells in the well (can be the same part as the dispensing unit).

In certain specific aspects, the automated system or device comprises a multi-well plate. The multi-well plate can comprise a transwell insert. In another example, the multi-well plate does not include a transwell insert. The multi-well plate is configured such that the cells can be exposed to more than one treatment cycle and such that the location of individual cells does not change over the different treatment cycles. It should be understood that each “unit” can be distinct or combined with another unit if the device, for example, the washing unit can be part of the dispensing unit. In additional aspects, the multi-well plate is configured such that the cells can be exposed to more than one treatment cycle and such that the relative location of individual cells does not significantly change over the different treatment cycles.

In certain aspects, the system or device described herein further comprises a holder, platform, or stand for the plate. Optionally, the holder or stand comprises a conveying unit for conveying the multi-well plate to engage with one or more of the dispensing unit, the optical imaging unit and the washing unit. In some embodiments, the holder, platform, or stand remains in a fixed location during the cycling. In such aspects, the dispensing, imaging and washing occur while the plate remains at a fixed location. In other examples, the holder or stand moves to different modules or units during the cycling.

The optical imaging unit described herein can further comprise a microscope, for example, a fluorescence microscope. In certain embodiments, the optical imaging unit (e.g., a fluorescence microscope) is located under the multi-well plate and can optionally be configured to move from well-to-well. In other examples, the optical imaging unit is located under the plate and the plate moves such that different wells are over the optical imaging unit.

The dispensing unit is configured to dispense analyte fluid onto the plate. For example, the dispensing unit can be configured to dispense different analyte fluid into each well. In some embodiments, the dispensing unit comprises nozzles and/or pipette tips.

The washing unit can comprise one or more aspirators configured to aspirate the analyte fluid. The washing unit can, for example, be configured for laminar wash. In some examples the holder or stand is configured to tilt (e.g, rotatably coupled to a fulcrum) and the washing unit aspirates or otherwise displaces fluid when the plate is tilted. As discussed above, the washing unit can be integrated into the dispensing unit.

In some aspects, dispensing and washing units comprise tubing, connectors, manifolds, pumps, syringes, vacuum systems or connections to the vacuum systems. In some aspects dispensing and washing units can be the parts of a single system or have shared parts.

The system or device can additionally comprise a processor in electronic communication with the optical imaging unit; and a non-transitory computer-readable medium accessible to the processor and having stored thereon instructions that, when executed by the processor, cause the processor to receive or record the optical signal. In certain aspects, the system or device comprises one or more processors configured to dispense the analyte fluid, detect the optical signal and/or remove the candidate agent. In a further aspect, the system comprises a control module with a user interface allowing the operator to execute a method of screening for cell response, for example, a method as described herein. The system or device can additionally further comprise one or more of a system of storage and submission of the new analytes of the screening library with optional temperature control; and a liquid handling device capable of managing the tips, liquids, moving the plates or other vessels with specimens or analytes, connected to all other system components, and capable of processing one or more vessels (for example, plates) with specimens.

The invention also encompasses a non-transitory computer-readable medium storing the computer-readable instructions which, when executed by one or more processors, manages a method described herein.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows the principle of the method of treatment cycling. Cell cultures are shown and can include more than once cell type with some cells having unique features. The probability of a response can be low and treatment cycling allows the screening of multiple treatments with the same sample. The method allows research into cellular phenotypes (e.g., CD4/CD8, etc.) and/or confirmation of long-term responses such as activation-induced markers, cytokine expression, etc., for example, by surface antibody staining (e.g., using a labeled antibody) of fixed cells using the same automated device. Data analysis can overlap with long-term/final readout (shown as a small dark “X” at the bottom of the figure) and quick responses related to certain cycles (shown as white circles) of the same cells). In more detail, the figure shows that cellular responses to a library of treatments (analyte fluids) are tested. The treatment exchange cycles show how many cells develop quick optical (fluorescent) readout associated with activation at each treatment cycle. Post-cycling, different downstream assays can be applied. In this example, AIMSpot is demonstrated. Post-cycling, the cells are incubated to develop long-term responses, and then stained via the membrane in automated manner with fluorescent antibodies either showing their activated phenotype (e.g., by detecting an AIM marker such as CD69, 4-1BB, OX40, or CD40L, or a combination thereof) or cytokine production.

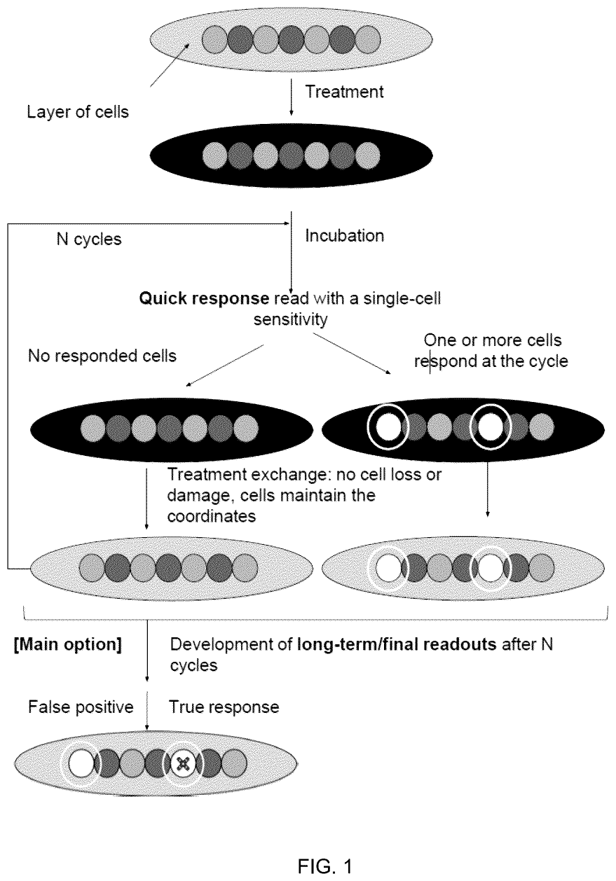

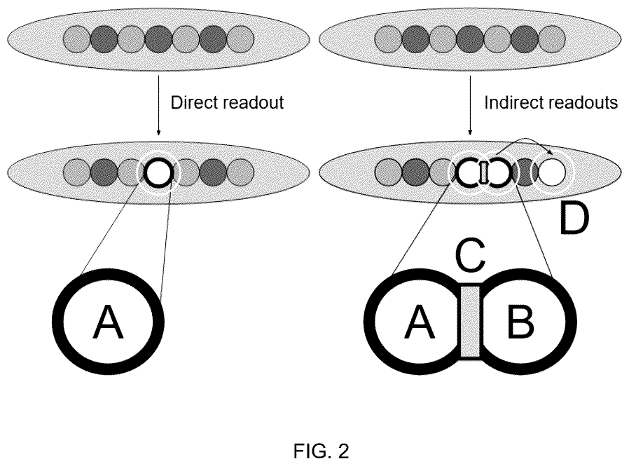

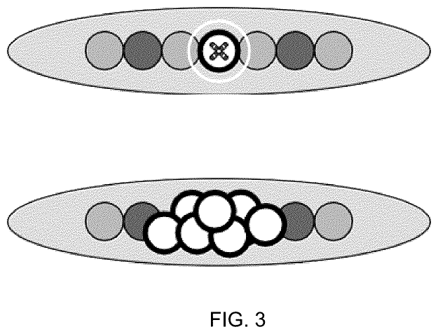

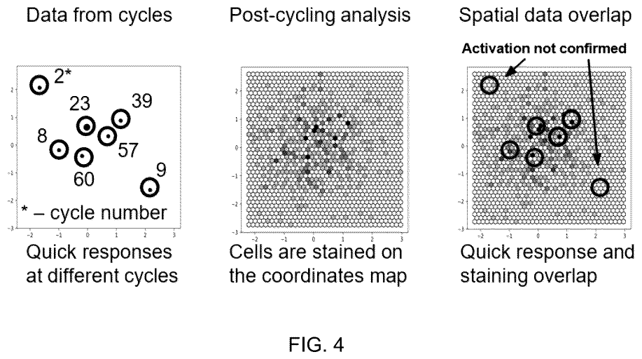

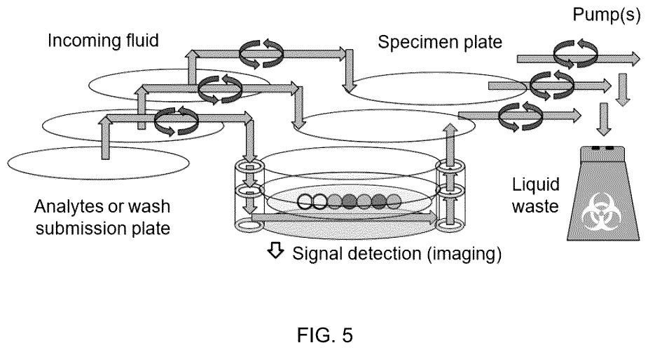

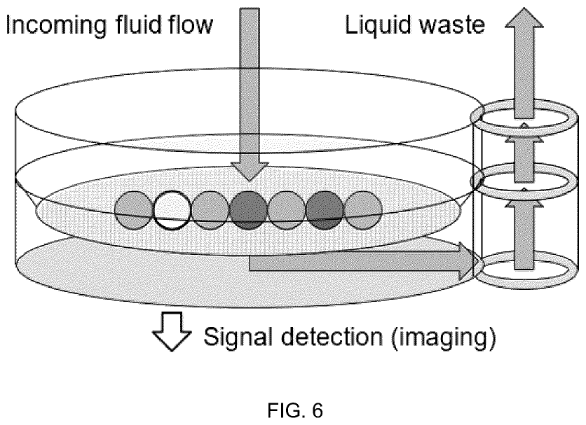

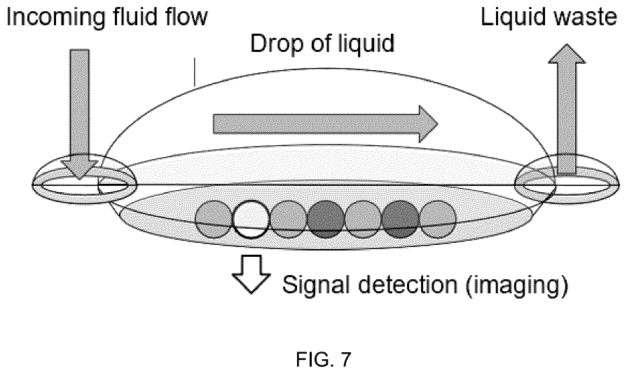

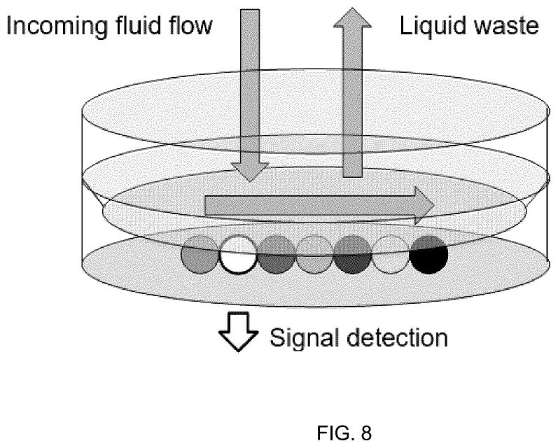

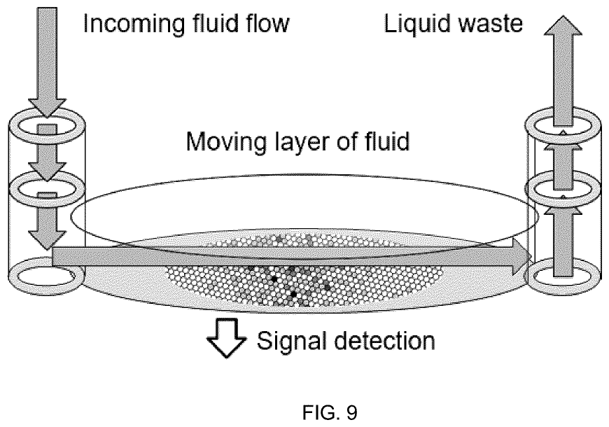

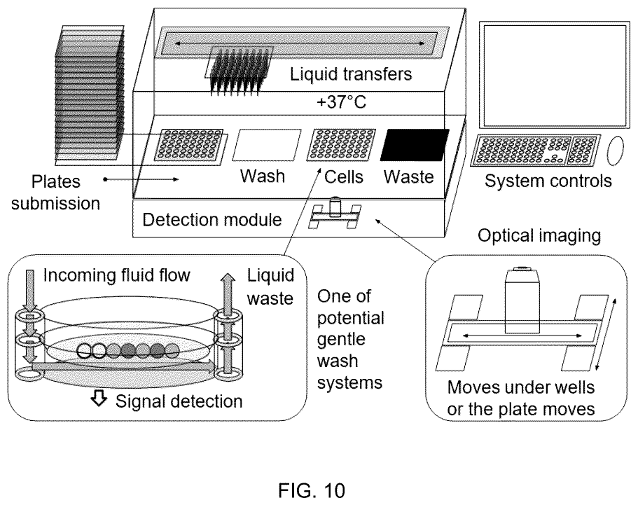

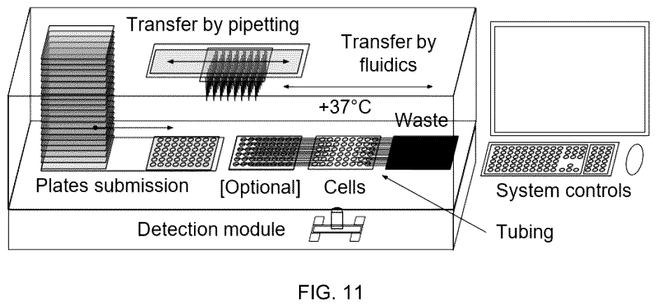

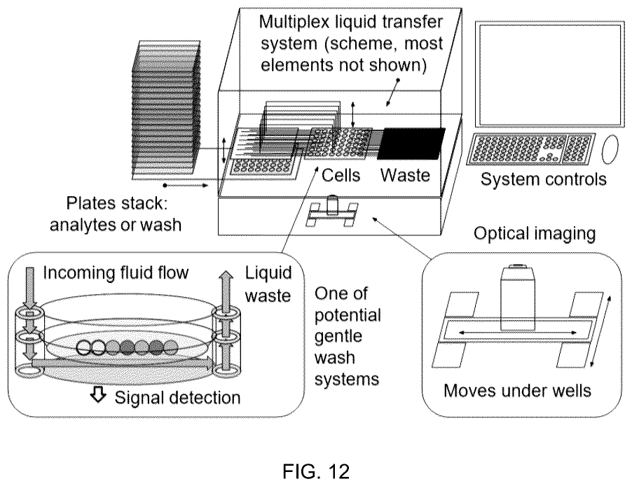

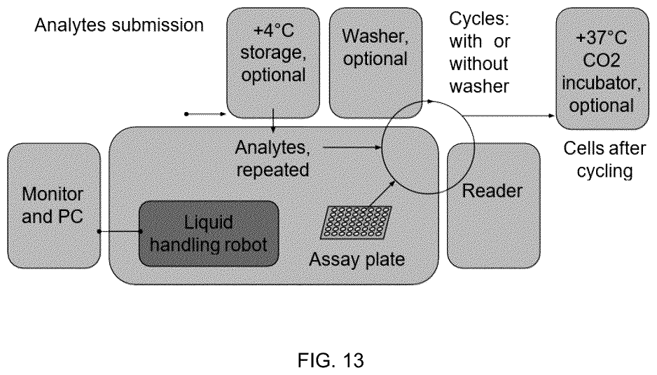

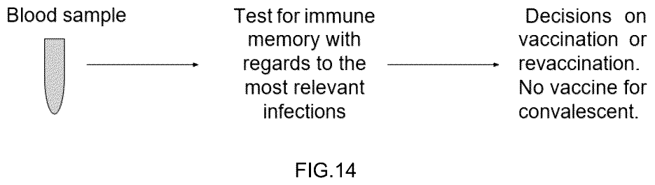

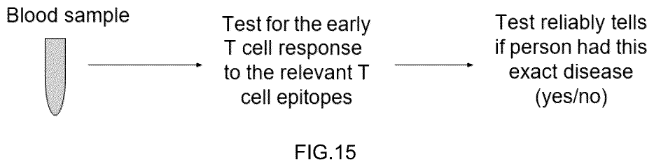

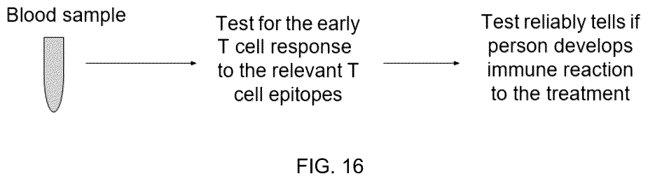

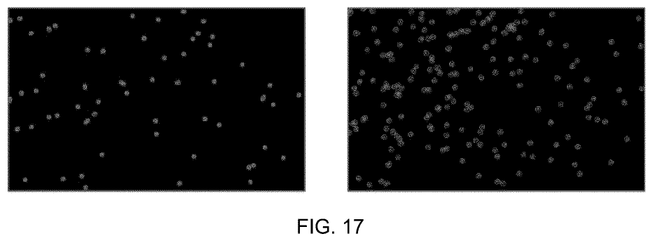

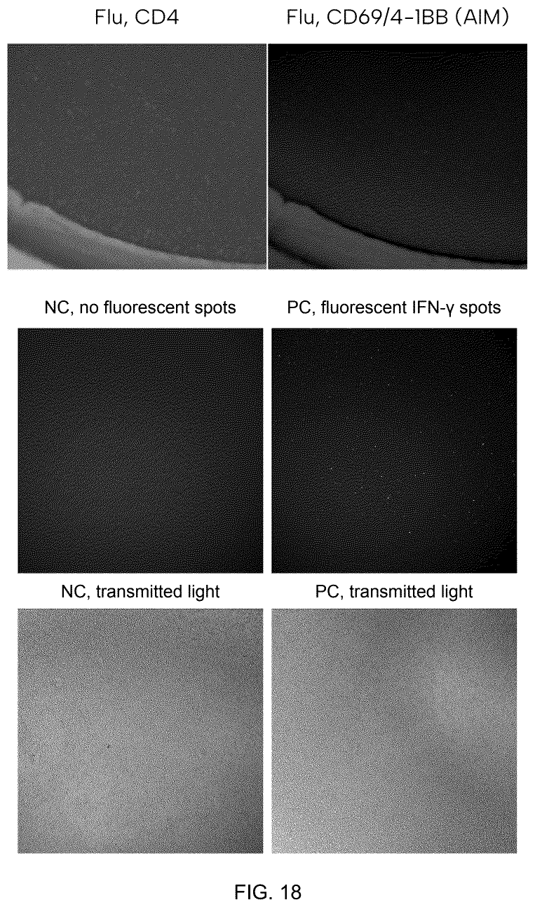

The identical treatment can occur simultaneously or in one cycle in multiple wells of the multi-well plates or multiple alternative vessels, or different treatments can occur simultaneously or in one cycle in different wells. In another example, the same sample (or samples) can be distributed for different treatments and/or the same treatments can be given in different wells in a different order. Cells are shown as a monolayer. In different embodiments, they may form multiple layers or represent the non-dissociated tissue. Layers can, in turn, be separated by the filtration mesh (as discussed elsewhere) or held together by different means, comprising cross-linked gels, bispecific antibodies, and fibrous proteins. Magnetic microbeads bound to the cells can, in some embodiments, can assist the layers'formation and stabilization. In one embodiment, the sample is pre-treated to provide a fluorescent readout. However, the sample or part of the mixed sample cells can be preliminarily separated, differentiated, and pre-treated to obtain optimal readouts. Pre-treatment schemes may comprise treatment with small molecules and different biologics, including antibodies, nucleic acids, and their analogs, together with the delivery vehicles, viruses, and pseudoviruses. In certain embodiments, these additional treatments may be given along with the analytes or between the cycles when the analytes are added.

FIG. 2 shows the principle of registering the direct (A) and indirect (B, C, D) responses. The embodiment where the T cell becomes fluorescent after activation represents the situation of the direct detection of the activated cell (A). In other embodiments, the signal of the T cell can be read indirectly via detecting the formation of the immune synapse (C) or the signal coming from the cell that presented the antigen and activated the T cell (B). These signals can, for example, comprise the detection of the cytoskeleton or mitochondrial network restructuring and multiple other events that occur in the cell that presents the antigen and actively interacts with the T cell. The detection of these changes may be fluorescent or rely on other optical detection methods. Another indirect T cell activation detection method is (D) detecting changes in the neighboring cells that follow the T cell activation and downstream signaling. For example, part of the cells in the sample may be responsive to the T-cell cytokines and generate detectable readouts. In some examples, the readouts can rely on responder cells. In additional examples, the readouts can be optical or based on other methods.

FIG. 3 shows the alternatives for downstream workflows after the treatment cycling. It depicts some of the potential downstream assay alternatives. It is important to note that in the case of T cell activation, researchers usually are interested in the T cell phenotypes and types of activation. The downstream assays may provide these data. The cycling assay itself may include cycles of the surface and intracellular staining with antibodies and cell fixation, providing the sample preparation for downstream applications.

Downstream workflows can include identifying cell features related to a response. Downstream workflows are optional but may help in data cleaning and enrichment. In one example, the downstream workflow includes one or more of the following optional steps, and can include others:
1. AIMSpot: cells are stained for surface markers at their relative positions, fixed and stained for intracellular proteins.
2. Cell culture can be sorted for select cell types/response.
3. Cell culture can be left for proliferation/death/other events.
4. Cell culture medium can be taken for biological testing.
5. Cell culture can be stained for flow cytometry or stained with barcoded antibodies/tetramers for downstream sequencing.
6. Cell culture can be used for bulk or single-cell sequencing.
7. Relative location of cells is fixed for spatial transcriptomics.
   For example, if the response is proliferation, use of an expanded clone has multiple analytical advantages.

FIG. 4 illustrates the principle of spatial data overlap using AIMSpot (cycles and post-cycling). The dynamic data of yes/no activation responses can be potentially contaminated with false-positive readouts (originating from irrelevant cell activation events, for example, initiation of apoptosis). Th dynamic yes/no response data, due to the maintenance of cellular coordinates during multiple treatment exchange cycles, can be finally overlapped (juxtaposed) with the post-cycling data of in situ antibody staining of the same cellular layer. Real activation of the cells can be confirmed by AIM marker expression or cytokine production, or a combination thereof. More complex studies are possible when T cell clones are grown and spatial sequencing or TCR sequencing are applied.

The figure shows that individual cell locations/coordinates are traced. Downstream analysis can include growing T-cell clones and transcriptome analysis. T-cell clones can, for example, be picked by automated colony pickers for sequencing of T-cell receptors (TCR). This combination of methods can give up to 1000-fold and higher increase efficiency in sample use for TCR discovery.

FIG. 5. shows one example of fluid exchange applied substantially simultaneously in multiple wells. The figures shows cells in transwell inserts. As shown in the figure, the liquid is transferred by pumps via the tubing. Treatment exchange takes place under the membranes of the transwell inserts. The pump liquid transfer system can be connected by tubing to a plate. Each well contains a transwell insert with a membrane at the bottom of the insert. Cells are layers on the membrane. One liquid transfer system removes previous treatment from below the membrane while a new wash/analyte/staining liquid is added.

FIG. 6 is a schematic showing a second example of fluid exchange in a single representative well of a multi-well plate. In this example of treatment exchange, cells are perfused on the membrane and layered on the membrane of a transwell insert. The treatment includes peptides and/or peptide pools corresponding to antigens. The readout is fluorescence of activated T-cells by fluorescence microscopy. For the treatment exchange, liquid can be slowly added inside the transwell insert on the top of the perfused cells and taken from the bottom of the chamber. Separation principle: mesh filtration; risk of disturbance exists. Signal detection: From the bottom to the top relative to the cells, in the same unit or a separate unit.

FIG. 7 is a schematic showing a third example of fluid exchange in a single representative well of a multi-well plate. In this example of treatment exchange, there is laminar flow over immobilized cells and the cells are immobilized at the bottom of a flattened well. The treatment is peptide libraries corresponding to common vaccines. The readout is immune synapse formation by fluorescent/optical microscopy. Treatment exchange is by laminar flow. Separation principle: gel or adhesion, non-disturbing shear flow. Signal detection: From the bottom or top relative to the cells, in the same or a separate unit.

FIG. 8 is a schematic showing a fourth example of fluid exchange in single representative well of a multi-well plate. In this example of treatment exchange, there is flow over the membrane which is over the cells, and the cells are adherent cells with a reporter system for pathway activation. The treatment is a large drug library with a low probability of positive response and every drug can be tested at increasing concentration. The readout is fluorescence/luminescence/death, etc. of the activated cells. Treatment exchange is carried out over the membrane without disturbing the cells. Separation principle: mesh shielding with cells on the bottom of the well. Signal detection: From the bottom or top relative to the cells, in the same or a separate unit.

FIG. 9 shows a fifth example of fluid exchange in single representative well of a multi-well plate. In this example of treatment exchange, cells are inside the microwell/microgrid array and cells are layered on the flat-bottom microwells/grid inside the well. The treatment is peptides or peptide poos corresponding to antigens. The readout can be immune synapse formation by fluorescent/optical microscopy. For treatment exchange, a layer of fluid exchanged over the microwells. Treatment exchange can be conducted by pipetting, fluidics or a combination thereof. Separation principle: cells are protected from flow by the walls of microwells/grid. Signal detection: From the bottom or top relative to the cells, in the same or a separate unit.

FIG. 10 shows one of the treatment cycling device example wherein the vessel with the specimens has a fixed location during the treatment cycles. This approach is favorable since the cellular locations/coordinates are important to trace through the assay. The liquid transfer principle is pipetting only. Liquid transfer can be conducted by pipetting only (e.g., a liquid handler robot). In this embodiment, cells do not change their locations until they are removed.

FIG. 11 shows another treatment cycling device example wherein the vessel with the specimens has a fixed location during the treatment cycles or the vessel on the stage of the microscope/imager can move for imaging (to allow having a stable optical system). This approach may be less favorable in certain situations since the cellular locations/coordinates are important to trace through the assay, however if the movements are slow they can be managed; the plate must stay as installed through all the cycles. The liquid transfer principle is hybrid and comprises both pipetting system and fluidics system. Fluidics systems can be based on a combination of multiple peristaltic, diaphragm, vacuum, or other pumps. If the connecting tubes are flexible, the stage holding the cell vessel (for example, plate) can move. Special plate design with liquid inlets and outlets can simplify the tubing connection. In this embodiment, cells do not change their location until removed. The detection module can be stable and the plate can move if tubing is flexible. Pipetting can be conducted to the intermediate transfer plate or direction on the test plate.

FIG. 12 shows yet another treatment cycling device example wherein the vessel with the specimens has a fixed location during the treatment cycles. The liquid transfer principle is only a fluidics system (no pipetting). A liquid exchange system can be organized below the wells or around the wells, to allow optical detection from the top of the plate and from below the plate. All potential embodiments are not shown. A substantial difference in this example is the lack of the pipetting by liquid handling robotics which is replaced by flow manifolds powered by peristaltic, diaphragm, vacuum, or other pumps. In this example, the cell do not change their location until they are removed.

FIG. 13 shows a further treatment cycling device example (based on multiple separate units) wherein the vessel comprising the specimens is moved automatically between the modules during the treatment cycles. This approach allows for building and programming an automated set of third-party equipment available on the market to create the treatment cycling workflow. Though the plate in this embodiment is moving, this approach also allows for specific cellular locations or coordinates to be traced throughout the assay by providing smooth plate moves, cellular immobilization by attachment to the plate or membrane, or cellular embedding in the gel. Cells are stable in the plate that is moving between the units. In this example, cells in plate are transferred between equipment units. Plate transfers can be improved if the cells are attached or gel-embedded.

FIGS. 14-16 show the potential uses of methods described herein for studying the vaccination status and vaccine efficacy, studying the spread of infections, studying the immunogenicity of cell and gene therapies that bring potential T cell antigens inside the organism. The advantages are the use of a minimal sample volume and high sensitivity compared to conventional approaches. Researchers will be able to use lower sample volumes to detect the response; alternatively, they may be using the same sample volume but obtain more data from the sample (epitope mapping or multiplex responses).

FIG. 14 illustrates a process for vaccine/infection protection status; the required sample volume is minimal for the same amount of data.

FIG. 15 illustrates a process for pandemic response preparedness. Time after infection for the test validity is minimal due to high sensitivity. The earliest potential time point is 2-3 days post contact. The cost of the test is much lower than the T-cell receptor sequencing-based analogs.

FIG. 16 illustrates a process for cell and gene therapies non-immunogenicity. Time after treatment for the test validity is minimal due to high sensitivity. The earliest time is 2-3 days post treatment. The cost of the test is much lower than the sequencing-based analogs. More applications: testing the effects of tolerizing and therapeutic, cancer vaccination, and many more.

FIG. 17 are fluorescence microscopy images of cells among human PBMCs demonstrating Ca²⁺ flux. The left image shows cells among human PBMCs monolayer demonstrating Ca²⁺ flux after 2 cycles of treatment (no treatment, Flu peptide pool), one field of view with number of activated cells n=62 (cells brighter than the pre-set threshold are counted using QUpath). The image on the ring shows the number of cells among human PBMCs monolayer demonstrating Ca 2+ flux after 3 cycles of treatment (first 2 cycles+Covid peptide pool), one field of view with number of activated cells n=183. Previously activated cells are not excluded from analysis.

FIG. 18 are fluorescence microscopy images. Top two images: T cell markers can be stained 24 hours after the Ca++ signaling data is collected (Ca²⁺ sensor signal photobleached before staining). Human PBMCs were stimulated with Flu peptide pool and cellular phenotypes were studied; staining was performed through the transwell membrane. For the data analysis images can be overlapped. Bottom images: Intracellular staining of the IFN-γ “spots” to confirm T-cell activation, Negative control (NC) vs positive control (PC) readouts; cell densities are demonstrated in the transmitted light images.

### DETAILED DESCRIPTION OF THE INVENTION

As used herein, the words “a” and “an” are meant to include one or more unless otherwise specified.

The term “about” as used herein, in reference to a numerical value or range, allows for a degree of variability in the value or range, for example, within 20%, within 10%, within 5%, or within 4%, or within 2% of the value or range, depending on the context.

A “specimen,” or “biological sample” as used herein, is a biospecimen or biological sample obtained from experimental animals, humans, in vitro cell lines, or other sources. An essential feature of the specimens described here is that they comprise live and functional cells capable of responding to diverse treatments. Non-limiting examples of biological samples include tissue, fluid, and other sample taken directly from a subject, as well as sample resulting from one or more processing steps, such as separation, centrifugation, genetic engineering (e.g., transduction with a viral vector encoding a recombinant chimeric receptor), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine, saliva sweat, and tissue and organ sample (e.g., sample from a tissue or organ containing a tumor), including processed sample derived therefrom. In some embodiments, the biological sample is a biological fluid sample or a biological tissue sample. In some embodiments, the biological sample is a biological tissue. In some aspects, the biological sample from which the immune cells are derived or isolated is blood or a blood-derived sample, or is derived from an apheresis or leukapheresis product. When blood is used as the biological sample, an anticoagulant such as heparin, citrate or ethylenediaminetetraacetate may be added to the blood, as necessary. A fraction containing a T-cell and an antigen-presenting cell prepared from blood by a conventional method may be used as the biological sample.

A “cell culture” is the growth of live cells under controlled conditions. The term “primary cell culture” implies that the cell culture is derived from a live organism in contrast to immortal cell lines also studied in vitro. In further aspects, the biological sample is dissociated tissue culture, primary cell culture, cell line, or mixtures of thereof. In certain specific aspects, the biological sample is a mixed culture of T-cells and antigen-presenting cells. The cell culture can, for example, be adherent, partially adherent, non-adherent or suspension culture. The biological sample or cell culture can be pre-conditioned by a variety of methods including, but not limited to, mechanical or enzymatic dissociation, centrifugation, magnetic or other tools for specific cell types, isolation, separation, enrichment or depletion, freezing/thawing, pre-treatment or incubation at different culture conditions, transfection and other genetic modifications.

The term “sample” as used herein can be used to refer to a specific portion of the specimen, biological sample, or cell culture on the solid support. For example, when the support is a 6-well plate, the plate can be described as having a sample in each well. The sample in each well can be derived from the same biological sample or from different biological samples. In certain specific aspects, the sample is a cell culture.

The terms “analyte fluid,” “analyte composition,” “analyte liquid” or “analyte solution” are used interchangeably herein to mean a liquid composition comprising a candidate agent. The candidate agent can, for example, be a small molecule or a biologic. A non-limiting example of the analyte fluid is the peptide diluted in DMSO and further diluted in the optimized T-cell culture medium.

The “candidate agent” is the agent being tested or screened for biological response. Non-limiting examples of candidate agents are small molecule drug candidates, peptides, antibodies, and libraries of any of thereof. A “candidate library” or “candidate agent library” is a mixture of 2 or more (usually up to several hundred) individual candidate agents. The “candidate agent” can, for example, be a potentially activating agent and/or that is added from outside the cell.

“Single-cell sensitivity,” as used herein, means that even if one single cell responds to treatment, the method or system can discriminate this response from the background. In certain aspects, the treatment cycling systems and methods described herein have single-cell sensitivity.

The terms “transwell system,” “well insert,” “transwell insert,” and “culture inserts,” are used interchangeably herein, to refer a product that is semi-permeable to air and liquid and that is placed inside the regular wells or “outer wells” of a multi-well cell culture plate or other vessels. These outer wells can have a design that allows for ease of medium/liquid addition, removal, or exchange. The “well insert” can be referred to as a “top chamber” or “upper chamber” and the multi-well plate or other support, or well or reservoir thereof, can be referred to as the “bottom chamber.” In some embodiments, these inserts allow cells to migrate via the semi-permeable membrane or mesh at bottom of the insert. In other embodiments, the membrane or mesh at the bottom of the insert is not permeable to cells.

As used herein, a “mesh membrane” or a “mesh,” as used herein refers to a semi-permeable or permeable membrane, having an open weave with openings through which liquids can pass, formed as a woven material, a perforated material or the like. In certain aspects, the mesh is a nylon mesh. The opening or pore of the mesh can be from 30 um to 1 mm. In certain aspects, the openings or pores of the mesh can be about 0.1 um to 5 um in size.

“Laminar cell wash,” as used herein, is a method to exchange the liquid component of the cell suspension or cultured monolayer by creating a liquid shear flow that does not impact the attachment of the cells to the bottom of the cell culture vessel.

A “downstream workflow,” “downstream assay,” “downstream method,” or the like as used herein, is a method applied after the treatment cycling.

“Direct readout” is the direct detection of a cell response, e.g., by a change in their optical property.

“Indirect readout,” is the indirect detection of a cell response, e.g, their interactions with neighboring cells or the responses of a neighboring cell.

A positive readout indicates that the cell response (e.g., T-cell activation) was detected.

A negative readout (e.g., lack of T-cell activation) indicates that cell response was not detected.

The systems and methods described herein utilize treatment cycling. “Treatment cycling,” is the method described herein that allows repeating treatments of the same sample. Repeating treatments allow the detection of one or more events of low probability (different subpopulations of cells responding to the certain treatments) within the same sample, therefore making the process either more sample-efficient and/or more sensitive, e.g., for the users that prefer not to decrease sample volume. For some users, the method described herein can be simultaneously more sample-efficient and more sensitive. Alternatively, this process can be seen as recycling or re-use of the same samples in a multiplicity of experimental treatments. This process can also be described as re-running an experiment seeking a positive response until it appears. In some embodiments, a positive response can be obtained in multiple cells and/or samples at different treatment cycles. In certain aspects, when a sample is positive for a response (such as T-cell activation) in a cycle, that sample is not subjected to a subsequent cycle of different treatments. In yet another aspect, when a sample is either positive or negative for a cell response (e.g., T-cell activation) in a cycle, that sample still can be subjected to one or more subsequent cycles of different treatments. A positive response can, for example, be a fluorescent, luminescent, or other signals above the background from the cells, interaction of the cell with another cell in the sample, or its metabolic product, and/or change of the phenotypic features by optical imaging of the cell(s) of interest or the cell(s) interacting with the cell of interest or its metabolic products. The optical signal can be detected by microscopy, for example, fluorescence microscopy.

With respect to standard T-cell epitope mapping methods, most T-cell immunologists seek to answer a few questions: “Is the T-cell activated?” and if yes, then “How is the T cell activated?”, and “What cytokines does the T cell produces after activation?” or “What surface markers T cells are expressed after activation?” The methods utilized in the art aim to answer these questions in a single experiment. The system and method described herein can answer the question “Is the T-cell activated?” multiple times (depending on the number of cycles) more effectively using less samples. After treatment cycling, a downstream assay, e.g., AIMSpot, AIM assay, and/or other methods described herein or known in the art can then be used downstream of the treatment cycling, to further characterize the cell response or T-cell activation, e.g, the type of T-cell activation and sequencing (for example, nucleic acid sequencing of the TCR receptor).

The systems and methods described herein combine quick readouts with gentle wash/analyte exchange and/or treatment exchange. The gentle wash/analyte exchange and/or treatment exchange methods described herein avoid the use of high-speed centrifugation. In certain embodiments, the gentle wash/analyte exchange and/or treatment exchange methods described herein avoid the use of centrifugation. Centrifugation is almost universally utilized for washing cells. Centrifugation is difficult to incorporate in an automated system and method, and can negatively affect cell viability and quantity, can lead to the generation of cell aggregates and decrease cell's performance in the assays, especially when the centrifugation is run at normal-to-high speeds for cell culture (>250×g) and repeated multiple times. The present invention therefore utilizes methods and techniques that can be readily automated and do not lead to cell loss or damage. Treatment cycling can also utilize methods and techniques that maintain the cells'relative spatial locations or coordinates on the support, e.g., in the well or on the membrane.

The systems and method described herein comprise a cell culture system wherein imaging and treatment exchange are conducted while allowing tracking of individual cells without cell loss and without significant changes of relative cellular locations (e.g., a cell's location or position relative to other cells in the culture) and/or a cell culture system which allows detection of individual cellular features and responses over time. Optionally, the systems and method described herein also comprise downstream (post-cycling) staining and characterization of additional cellular features and markers in the same traceable cells and/or at their corresponding locations and/or on the same support as the treatment cycling; said post-cycling characterization can be done with live or fixed cells. For example, described herein are devices comprising a multi-well plate configured such that the relative location of individual cells does not significantly change over the different treatment cycles. In some examples of the methods and devices described herein, the relative locations of the cells does not significantly change if at least about 85%, 90% or 95% of cells in the sample retain their relative cellular location after 2 total cycles, after 3 total cycles, after 4 total cycles, after 5 total cycles, after 6 total cycles, after 7 total cycle, after 8 total cycles, after 9 total cycles, or after 10 or more total cycles. In certain specific aspects, the cells are non-adherent cells and the relative locations of the cells does not significantly change if at least about 85%, 90% or 95% of cells in the sample retain their relative cellular location after 2 total cycles, after 3 total cycles, after 4 total cycles, after 5 total cycles, after 6 total cycles, after 7 total cycle, after 8 total cycles, after 9 total cycles, or after 10 or more total cycles. Because treatment cycling as described herein retains the relative location of the cells, individual cells can be traced or monitored using imaging during the multiple cycles.

The treatment cycling approach ensures that cells are not exposed to a medium-free environment during cycling. Thus, the systems and methods described herein can provide cell culture conditions over hours, or days. In some examples, the analyte fluid of each cycle is different and multiple cycles are possible, for example, 5 cycles, 10 cycles, 20 cycles, 50 cycles, 75 cycles, or 100 cycles. In additional examples, the treatment schedule (e.g., the analyte fluid) can be different for different wells. The cycles of treatment exchange can be automated and/or standardizable, e.g., for multiple wells, and/or coordinated with the downstream assay, e.g., imaging. In some examples, at least 1 million cells are on the support.

In one specific example, the cells of a sample form a layer on top of a semi-permeable membrane of a transwell insert and the transwell insert is placed in a well of a multi-well plate. The membrane restricts or prevents the migration of cells from the transwell insert (e.g., from entering the bottom of the well of the multi-well plate). Treatment from a preceding cycle can be removed from the well of the multi-well plate without disturbing the cell locations and without exposing the cells to a medium-free environment. Addition of a new analyte fluid to the well of the microwell plate can diffuse and reach the cells in the insert. Removal of analyte fluid in any cycle can, for example, be performed by pipette and/or tubing in connection with a pump which can modulate the flow rate. Other methods of separating the cells from the treatment exchange or flow of the new analyte fluid are described, for example, in the figures. In certain aspects, the transwell insert is fixed or rendered immobile when placed in the well of the multi-well plate. In additional examples, the opening of the well and/or transwell insert is covered to prevent or decrease evaporation.

When the treatment cycling approach is combined with the multiple signals per vessel/well/microchip for the spatial transcriptomics, i.e., Cycle-Location Barcoding (CLB), the efficacy of the specimen use, for example for determining TCR sequences and activation types, may be additionally increased up to 10-fold or more. Applying treatment cycling and CLB together can increase assay efficacy up to 1000 times or more.

As described herein, each treatment cycle includes a response readout step (detection of the cell response or detection of T-cell activation) and a removal step wherein the analyte fluid is removed. The removal of the analyte fluid removes all or a portion of the analyte fluid from the solid support (e.g., the multi-well plate) and thus removes the candidate (potentially activating) agent but retains the sample comprising the cells on the solid support (e.g., the cell culture). “Gentle analyte removal,” treatment exchange, “gentle analyte removal/treatment exchange,” and the like as used herein, refer to a set of methods that allow removal of the analyte fluid from a sample without extreme stress or damage to the live cells, preferably preserving their capability for a functional response and/or their relative locations on the support or on the bottom of the well or the membrane. In certain aspects, the gentle analyte removal and/or treatment exchange preserves a cell's ability for a functional response and its relative location. In certain aspects, the removal of the analyte fluid and/or treatment exchange is gentle enough that the cell viability and/or the cell quantity of the retained sample is decreased by no more than about 5% after 10 total cycles. The methods used for removal and/or treatment exchange do not include high-speed centrifugation. In certain aspects, the method used for removal of the analyte and/or treatment exchange does not include centrifugation. Standard centrifugation at high centrifugal forces is not considered a gentle cell wash since it can lead to cell loss, death, or dysfunction. In certain aspects, the gentle analyte removal and/or treatment exchange can include analyte exchange wherein previous potentially T-cell activating analyte solution is removed, and the next potentially T-cell activating analyte solution is added. In additional aspects, the removal step can comprise a gentle wash which, for example, is a wash step wherein a wash buffer with no components to activate the cells is introduced to the sample. An example of a wash buffer for T-cell activation cycling is a T-cell medium. The gentle wash and/or treatment exchange can be performed at the optimal physiological temperatures, ideally at +37° C.; however, temperature variation should be possible without affecting the assay. In certain aspects, the temperature is no lower than +20° C. and no higher than +40° C. Ideally, the gentle wash and/or treatment exchange will retain the cells in the same 2D orientation in the well. For some assay implementations these stable cellular coordinates will be critical.

As described above, the invention encompasses detecting a cellular response to a candidate agent (e.g., a potentially activating agent), the method comprising:
i. a first cycle comprising:
   a. contacting a sample comprising cells with an analyte fluid, wherein the sample comprising the cells is on a solid support and wherein the analyte fluid comprises a candidate agent in a liquid medium;
   b. detecting the cellular response by detection of an optical signal in the sample on the solid support; and
   c. removing the analyte fluid from the solid support thereby removing the candidate agent while retaining the sample comprising the cells on the solid support;
ii. a subsequent cycle comprising:
   a. contacting the retained sample comprising the cells of the preceding cycle with a new analyte fluid, wherein the new analyte fluid comprises a new candidate agent in a liquid medium, wherein the new candidate agent is different from the candidate agent of the preceding cycle;
   b. detecting the response by detection of an optical signal in the retained sample on the solid support; and
   c. removing the analyte fluid from the solid support thereby removing the candidate agent while retaining the sample comprising the cells on the solid support;

repeating the subsequent cycle at least N times, wherein N is an integer greater than or equal to 0, wherein each new analyte fluid of each subsequent cycle comprises a different candidate agent from that of the preceding cycle. The analyte fluid is removed by a gentle analyte removal method or treatment exchange method as described herein. In certain aspects, the removal of the analyte fluid or treatment exchange is a method whereby the cell viability and/or the cell quantity of the retained sample is decreased by no more than about 5% after 10 total cycles and that does not comprise centrifugation. In certain examples, the detection of an optical signal in a cycle comprises more than round of detection before analyte removal or treatment exchange and the subsequent cycle. For example, a cycle can include multiple imaging or detection steps conducted over time, e.g., seconds, minutes, or hours apart.

The solid support can be any support on which cells can be cultured, including, but not limited to a cuvette, a tube, a capsule, a microtiter plate (also referred to herein a multi-well or multi-well plate) with microtiter wells, such as a 6-well plate, 12-well plate, 24-well plate, 96-well plate, 192-well plate a 384-well plate, 1536-well plate, or a membrane surface of a transwell inserts within these multi-well plates. Another example of a solid support is the “wall-less” plate sold by Curiox and/or described, for example, in US20140235468A1; the contents of which are expressly incorporated by reference herein. An additional example of a solid support is the plate with the bottoms of wells containing microwell or microgrid arrays for cell culture (similar to ones described in U.S. Pat. Nos. 9,068,155B2, 11,969,702B2, US20230407223A1), where microwells or microchambers prevent liquid flow in proximity to the cells at the bottom, but allow the treatment exchange within the microwells/microchambers via diffusion, when the liquid is exchanged in the larger well or the whole vessel. The solid support can be formed from any suitable materials, including, but not limited to, polystyrene or polyvinyl, or a derivative thereof. The solid support can also be a microfluidic device.

Treatment cycling includes the first cycle and at least one subsequent cycle. N is the number of cycles after the second cycle. Thus, when N is 0, there are two total cycles. In another example, when the total number of cycles is 10 this means that there is a first cycle and 9 subsequent cycles (or in other words, N is 8). In another example, when the total number of cycles is 100, this means that there is a first cycle and 99 subsequent cycles. Each subsequent cycle contacts the sample of the preceding cycle from which the analyte fluid was removed. The preceding cycle is the cycle directly before the cycle being referenced, for example, when the total number of cycles is 3, the first cycle is “the preceding cycle” for the second cycle and the second cycle is “the preceding cycle” for the third cycle. In another example, when the solid support is a 96-well plate wherein each well includes a sample and the total number of cycles is 3, analyte fluid can be removed from each well at the end of first cycle (while retaining the sample comprising the cells in the well), and that retained sample is treated with the new candidate agent in the second cycle. Next, analyte fluid can be removed from each well at the end of the second cycle (while retaining the sample comprising the cells in the well), and that retained sample is treated with the new candidate agent (different from the candidate agent of the second cycle) in the third cycle.

The analyte fluid can be removed from the solid support (e.g., the multi-well plate) by any method that displaces all or a part of the analyte fluid. For example, the analyte fluid can be aspirated or otherwise removed such that cells are retained in the well with minimal loss in viability and/or function (such methods can include tilting of the plate and/or use of a mesh to cover the cells). The analyte fluid can, for example, be removed using a pipette and/or tubing. In a further method, fluid (e.g., a buffer, other wash fluid or the new analyte fluid) can be added to the solid support thereby removing the analyte fluid by displacement as liquid waste (treatment exchange without washing). The new analyte fluid can, for example, be added to the solid support thereby removing analyte fluid of the preceding cycle, for example, using a pipette and/or tubing in connection with a pump which can modulate the flow rate.

In some examples, the solid support is a transwell insert (upper chamber) used with a multi-well plate (the bottom chamber) and a sample comprising T-cells is in each well of the insert. When such transwell inserts are used, the analyte fluid can be removed or displaced from the wells of the multi-well plate. Such a method is illustrated, for example, in FIG. 4. In yet another example, a mesh is placed over the cells and the analyte fluid is removed from the top of the plate. Such a method is shown, for example, in FIG. 6. In yet a further example, the cells are on top of a mesh or sandwiched between two layers of mesh and the analyte fluid is removed from under the mesh. Such a method is shown, for example, in FIG. 7.

In a further example, the analyte fluid is removed by laminar wash. Laminar wash devices are sold by Curiox (curiox. com) and have been described, for example, in WO2020028406A1 and U.S. Pat. No. 9,557,318B2; the contents of which are expressly incorporated by reference herein. In a laminar wash method, buffer or other wash fluid is dispensed and aspirated at opposing locations at a precise rate resulting in laminar flow with high flow rate at the top of the well and a low or static flow rate where the cells are. A laminar wash method is illustrated, for example, in FIG. 5.

In certain specific aspects, the invention is directed to a method of identifying or detecting T-cell activation, the method comprising:
i. a first cycle comprising:
   a. contacting a sample comprising T-cells with an analyte fluid, wherein the sample comprising the T-cells is on a solid support and wherein the analyte fluid comprises a candidate agent in a liquid medium;
   b. detecting T-cell activation by detection of an optical signal in the sample on the solid support; and
   c. removing the analyte fluid from the solid support thereby removing the candidate agent while retaining the sample comprising the T-cells on the solid support;
ii. a subsequent cycle comprising:
   a. contacting the retained sample comprising the T-cells of the preceding cycle with a new analyte fluid, wherein the new analyte fluid comprises a new candidate agent in a liquid medium, wherein the new candidate agent is different from the candidate agent of the preceding cycle;
   b. detecting T-cell activation by detection of an optical signal in the retained sample on the solid support; and
   c. removing the analyte fluid from the solid support thereby removing the candidate agent while retaining the sample comprising the T-cells on the solid support;

repeating the subsequent cycle at least N times, wherein N is an integer greater than or equal to 0, wherein each new analyte fluid of each subsequent cycle comprises a different candidate agent from that of the preceding cycle. The removal step is performed using a gentle removal, wash method and/or treatment exchange as described herein. In certain aspects, the removal of the analyte fluid (e.g., by treatment exchange or gentle wash) is a method whereby the cell viability and/or the cell quantity of the retained sample is decreased by no more than about 5% after 2 to 10, or 10 or more, total cycles and that does not comprise high-speed centrifugation. In certain aspects, the removal of the analyte fluid is a method whereby the cell viability and/or the cell quantity of the retained sample is decreased by no more than about 5% after 10 total cycles and that does not comprise centrifugation. In certain examples, the detection of an optical signal in a cycle comprises detection of multiple (e.g., at least two) different optical signals or multiple rounds of detection of the same optical signal before analyte removal and/or treatment exchange and the subsequent cycle. For example, when the method comprises multiple rounds of detection of the same optical signal, a cycle can include multiple imaging or detection steps conducted over time, e.g., seconds, minutes, or hours apart.

When the solid support is a multi-well plate, a microtiter plate, or any support comprising multiple discrete samples, each sample on the support can be contacted with a different candidate agent in a cycle or all cycles; for example, when all of the samples are from the same biological sample. In another example, each sample on the support can be contacted with the same candidate agent in a cycle or all cycles, for example, when some or all of the samples are from different biological samples. In certain aspects, one or more cycles, or all of the cycles, can include a positive control (an agent known to elicit a response in the cell; for example, an agent known to activate a T-cell). In certain additional aspects, one or more cycles, or all of the cycles, can include a negative control (an agent known to not elicit a response in the cell; for example, an agent known to not activate a T-cell).

Non-limiting examples of cells that can be used in the systems and methods described herein are cells of multicellular organisms, e.g., cells of invertebrates and vertebrates, such as myoblasts, neutrophils, erythrocytes, osteoblasts, chondrocytes, basophils, eosinophils, adipocytes, invertebrate neurons (e.g., Helix aspersa), vertebrate neurons, mammalian neurons, adrenomedullary cells, melanocytes, epithelial cells, and endothelial cells; tumor cells of all types (e.g., melanoma, myeloid leukemia, carcinomas of the lung, breast, ovaries, colon, kidney, prostate, pancreas and testes); cardiomyocytes, endothelial cells, lymphocytes (e.g. T-cells and B cells), mast cells, vascular intimal cells, hepatocytes, leukocytes including mononuclear leukocytes; stem cells such as hematopoietic stem cells, neural, skin, lung, kidney, liver and myocyte stem cells; osteoclasts, connective tissue cells, keratinocytes, melanocytes, hepatocytes, and kidney cells; induced stem cells and stem cell-derived cells. Suitable cells also include known cell lines, including, but not limited to, Jurkat T-cells, NIH3T3 cells, CHO, COS, etc.

In certain aspects, the cells or culture thereof comprise immune cells including, but not limited to, T-cells, B-cells and NK cells. In additional aspects, the cells or culture thereof comprise peripheral blood mononuclear cells (PBMCs). In additional aspects, the cells or culture thereof comprise T-cells. Such T-cells include, for example, CD4⁺, CD8⁺, and regulatory T-cells. A suitable example is a Jurkat cell. Jurkat cells are immortalized T lymphocytes first derived from the peripheral blood of a child with T-cell leukemia (Schneider et al., 1977, Int J Cancer 19 (5):621-6). The sample can comprise T-cells and APCs, such as professional APCs. Such professional APCs can be selected from the group consisting of dendritic cells, macrophages, monocytes and B cells.

In certain aspects, the candidate agent is a peptide or a peptide library or a disease-related antigen (e.g., a bacterial antigen, a viral antigen, or a cancer antigen). A candidate agent can be an agent from which the effect on T-cell activation and/or differentiation and/or modulation of other cell response is unknown. Candidate agents (or libraries thereof) include for example oligopeptides, polypeptides, proteins, antibodies, (peptide-)mimetics, and small molecules. In some examples, the candidate agent is screened for its ability to activate an immune cell. The candidate agent can, for example, be an infectious disease-associated candidate epitope, an autoimmune disease-associated candidate epitope, or a tumor-associated candidate epitope. An epitope refers to the portion of antigen that is recognized by B cells or T-cells, and the portion of the antigen to which an antibody binds. An epitope refers more specifically to the portion of antigen that is recognized by B cells or T-cells, and the portion of the antigen to which an antibody binds. An epitope is typically a small peptide of 2 or more amino acids (e.g., 2, 3, 4, 5, 6, 7, 8, 9, 10, 11, 12, 13, 14, 15, 16, 17, 18, 19, 20, 21, 22, 23, or 24 amino acids). Class I epitopes are typically about 8-15 amino acids in length, and more typically about 8-12 amino acids in length. Class II epitopes are typically about 8-24 amino acids in length, and more typically about 15 amino acids in length.

A biological sample or sample comprising T-cells can be stored in a suitable solution such that the T-cell and optionally, the antigen-presenting cell are maintained in a viable state. Examples of such a liquid include a medium generally used for culturing an immune cell in vitro. Such a medium is known, and examples thereof include MEM, DMEM, RPMI-1640, CTL-Test medium (Cellular Technology Limited), and the like. If necessary, the medium may be mixed with an additive such as fetal bovine serum (FBS) or L-glutamine. In a preferred embodiment, the biological sample or specimen is stored in a medium under conditions under which an immune cell can be cultured. Such conditions are known per se, and examples thereof include conditions at 37° C. in a 5% CO₂ atmosphere.

The solid support can be a microfluidic device. The candidate agent can be added to the microfluidic device comprising the cells or the candidate agent can be already present (e.g., pre-loaded) on the microfluidic device when the cells (e.g., as part of a patient's sample) are added. The microfluidic device can, for example, be made of glass, polymer or other suitable material. The flow of analyte fluid (fluid comprising the candidate agent) over the cells does not change the location of the cells. Analyte fluid can be removed using a gentle analyte removal or treatment exchange method described herein. In certain aspects, the methods include a gentle wash step. In other aspects, the methods do not include a wash step. The cells are retained on the microfluidic device after the gentle analyte removal and/or gentle wash or treatment exchange, and the cells can then be treated with a new analyte fluid (a new treatment cycle). In certain aspects, the T-cell activation treatment cycling is conducted using a microfluidic device comprising the T-cells.

The systems and methods described herein detect a cell response (e.g., T-cell activation) by detecting an optical signal in the sample on the support, for example, by fluorescence microscopy. In certain aspects, the optical signal indicates T-cell activation. For T-cell activation, the optical signal can, for example, indicate calcium flux, cytokine release, or the formation of a complex comprising a T-cell and an antigen-presenting cell (APC). In certain aspects, the optical signal is immune synapse formation, for example, a complex of a T-cell and an APC. In yet additional examples, the optical signal indicates calcium flux.

In certain aspects, the optical signal indicates calcium flux. Increased calcium concentration in the T-cell is a rapid and sensitive measure of cell activation. Thus, the optical signal can be provided by a calcium binding moiety (or a calcium probe) that includes a detectable label (e.g., a fluorescent label). A calcium binding moiety with a fluorescent label can be referred to as a “fluorescent calcium indicator” or a “fluorescent probe”, and can comprise a fluorescent dye coupled to a calcium chelator, and various products are commercially available. Examples of such fluorescent calcium indicators include Fura2, Fluo3, Fluo4, Indo1, Rhod2, and the like. The fluorescent probe may be protected with an acetoxymethyl (AM) group. Protection with an AM group imparts cell permeability to the fluorescent probe. Non-limiting examples of labeled calcium probes/indicators that can be used according to the methods described herein include x-Rhod-1 (a red fluorescent calcium indicator), Calbryte™ 630 AM (AAT Bioquest), Calbryte™ 520 AM (AAT Bioquest), as well as, Fluo-4, Fluo-Gold, Fluo-3, Fluo-2 available from Ion Biosciences. The labeled calcium probe can be added before, after or concurrently with the analyte fluid. For example, the calcium probe can be added to the cells before the analyte, incubated for about 1 hour and then it is washed off.

In additional aspects, the optical signal indicates cytokine release. Examples of cytokines secreted by activated T-cells include interferon gamma (IFN-γ) and interleukin-2 (IL-2). Accordingly, in some embodiments, the optical signal can be provided by a labeled moiety that binds to a cytokine. An example of such a labeled moiety is a fluorescently labeled anti-cytokine antibody, such as an anti-IFN-γ antibody or an anti-IL-2 antibody. The labeled moiety that binds to a cytokine can be added before, after or concurrently with the analyte fluid.

In yet further aspects, the optical signal is the formation of an immune synapse, in other words, a complex of a T-cell and an APC. Non-limiting examples for detecting the immune synapse are described, for example, in US20210356454A1 and Calvo et al. (2018), Front Immunol 9: 684; the contents of which are expressly incorporated by reference herein.

Representative labels that may be suitable for optical detection (e.g., associated or attached to an anti-cytokine antibody or calcium probe) include radioactive isotopes, fluorescers, chemiluminescence, chromophores, enzymes, enzyme substrates, enzyme cofactors, enzyme inhibitors, dyes, metal ions and metal sols. In some embodiments, the label is fluorescent.

Representative examples of non-proteinaceous fluorescent labels include allophycocyanins (trade name XL665); luminescent organic molecules, such as rhodamines, cyanines (e.g., Cy5), squaraines, coumarins, proflavins, acridines, fluoresceins, boron-dipyrromethene derivatives (commercially available under the tradename “BODIPY”), fluorophores known under the name “Atto”, fluorophores known under the name “DY”, compounds known under the name “Alexa”, and nitrobenzoxadiazole. The “Alexa” compounds are commercially available, e.g., from Invitrogen; the “Atto” compounds are commercially available from Atto-tec; the “DY” compounds are commercially available from Dyomics; and the “Cy” compounds are commercially available from Amersham Biosciences. Fluorescently labeled anti-cytokine antibodies are commercially available. Proteinaceous fluorescent labels may also be useful. Representative examples of fluorescent polypeptides include yellow fluorescent protein (YFP), cyan fluorescent protein (CFP), GFP, mRFP, RFP (tdimer2), and HCRED. Biotin-based labels may also be useful. Biotinylation of target molecules, including antibodies, is well known in the art. Biotinylated anti-cytokine antibodies may be detected by binding of a detectably labeled biotin binding partner, such as avidin or streptavidin.

In yet further aspects, the cells express a heterologous reporter gene and the expression of the gene provides the optical signal. Genetic reporters are used widely as indicators to study gene expression and cellular events coupled to gene expression for pharmaceutical and biomedical research. Typically, a reporter gene encoding the reporter is cloned into an expression vector that is then transferred into cells. Expression vectors can include viral vectors, plasmids, mRNA, and others. Following transfer, the cells are assayed for the presence of the reporter by directly measuring the reporter protein itself or the enzymatic activity of the reporter protein. Preferred reporters are those that can be identified easily and measured quantitatively when expressed in the effector T-cells. Many suitable examples are known to those skilled in the art, including fluorescent and luminescent reporters. Optionally the reporter is a bioluminescent reporter, such as a luciferase.

In some examples, a sample or cell(s) in a sample that exhibits a positive readout can be identified as exhibiting the cellular response, such as T-cell activation without the need for downstream assay. For example, when the optical signal is immune synapse formation, downstream assay may not be required for confirmation or characterization of T-cell activation.

A sample that exhibits a positive readout in one or more cycles can be further analyzed or characterized using a downstream assay. Such downstream workflow can include, for example, incubating the cells for several days (e.g, 5 or more days) and evaluating cell response (e.g., cell death, proliferation, cytokine production); collecting cell culture medium for analysis at different time points; sorting of the resulting cells of the specimen by cell type, activation state or other features using magnetic separation or other cell sorting methods; staining for flow cytometry or barcoded antibody staining for sequencing; determining the coordinates of the responded cells and applying an array of methods to perform the spatial transcriptomics for the 2-D specimen, optionally kept attached to the vessel bottom or transwell mesh or between two layers of the mesh; colony-picking by the third-party automated instruments; and/or bulk or single-cell sequencing of DNA or RNA, as the example sequencing of the T-cell receptor (TCR) mRNAs to find their sequence in parallel with the peptide specificity if the treatment included the peptides of peptide pools.

The downstream assay can, for example, determine the T-cell subtype and function of the positive sample. The T-cell type and function can be determined from expression of surface proteins (e.g. CD3, CD4, CD8, CD45RA, etc.) and cytokine production (e.g. interferon-gamma (IFN-γ), transforming growth factor beta (TGF-β), interleukin (IL)-2, IL-4, IL-17, etc.) by antibody-based methods such as flow cytometry, immunohistochemistry, or immunofluorescence, or by transgenic fluorophore expression. Downstream assays can, for example, include repeated cell activation or use in subsequent experiments alone or in the presence of other cells or tissue culture specimens.

In certain aspects, the downstream assay can be conducted on the same support as the treatment cycling. In addition aspects, the downstream assay is conducted by the same device that removes the analyte fluid.

As described above, the downstream assay can comprise staining and/or characterization of additional cellular features and markers in the same traceable cells or at their corresponding locations. Such post-cycling characterization can be done with live or fixed cells. The cellular features include phenotypic markers (such as CD4, CD8 and others), expressed cytokines or other secretory molecules captured within the cells (such as IFN-g, IL-2, TNF and others; perforin, granzyme B and others), expressed activation-induced markers (collectively called AIM, such as CD69, 4-1BB, OX40, CD40L and others). The use of FACS for the detection of activation-induced markers is described, for example, in Lemieux, Audrée, et al. “Enhanced detection of antigen-specific T cells by a multiplexed AIM assay.” Cell Reports Methods 4.1 (2024); the contents of which are expressly incorporated by reference herein. Contemporary methods of serial staining with sets of fluorescent antibodies allow further multiplexing of the final readouts. The main advantages of AIMSpot over ELISpot include cellular phenotyping, potential for high multiplexing of the data about activated cells; the main advantages over FACS-ICS/AIM is a higher potential to automating the workflow; high resolution data on antigen specificity (activation cycling).

In certain aspects, the downstream assay is an activation-induced marker (AIM) assay or a method that detects an activation-induced marker (or expression thereof). An “activation-induced marker” (AIM) is a marker that is expressed, or in which the expression is upregulated, after activation of a T cell. The AIM assay can detect any marker that is upregulated by a T cell upon activation of the T cell. In certain aspects, the assay comprises use of a labeled antibody, for example, a fluorescently labeled antibody, which binds to the AIM. Non-limiting examples of activation-induced markers include, for example, CD137/4-1BB, CD107, IFNγ, PD-1, CD40L, OX40, CD25, CD69, CD28, HLA-DR, CX3CR1, TIM3, LAG3, TIGIT, or a combination of any of thereof. In further aspects, the AIM is CD69, 4-1BB, OX40, and CD40L, or a combination thereof. In certain aspects, the downstream assay is flow cytometric activation-induced marker (AIM) assay. In certain aspects, the downstream assay is a method that detects an activation-induced marker and the optical signal (detected during cycling) indicates calcium flux, e.g., by adding a calcium binding moiety or a calcium probe to the that includes a detectable label.

In some aspects, the downstream assay is imaging of cells using a dye or a labelled antibody, for example. One specific method of a downstream (post-cycling) assay is staining or imaging of live or fixed cells with dyes or antibodies (e.g., a fluorescent-labeled antibody or an antibody comprising a fluorescent or dye label), that confirms or provides further characterization of the data or readout obtained during treatment cycling. In some embodiments, the staining or imaging is automated. Such methods are referred herein as “AIMSpot.” A non-limiting example of an AIMSpot method is one or more cycles of live human PBMCs treatment with candidate compounds that potentially activate the T cells. In this example the solid support is a transwell insert. All treatments of the cells in this example do not change the location of the cells, and all treatments are performed below the transwell insert membrane while the cells are cultured on the top of the membrane (including, for example, treatment with Brefeldin A, fixation/permeabilization buffer, blocking solution, staining solution, washing solution, cell storage and imaging solution). Post-treatment, the cells are given two hours to upregulate cytokine IFN-γ expression. Then IFN-γ secretion is blocked by treating the cells with Brefeldin A followed by 4 hours of incubation. After that, the cells are fixed in a buffer containing paraformaldehyde able to permeabilize the cellular membrane. Thereafter, the cells are treated with blocking solution containing serum and unstained antibody blocking FcG receptors. Then the cells are stained with fluorescent antibodies to human IFN-γ. Excess antibodies are washed off post-treatment. The cells are imaged using fluorescent microscopy. Small bright “spots” correspond to the cells producing IFN-γ, and the antigen specificity of these cells can be determined by the cycle number when the Ca++ flux was detected in them.

As described herein, one of the areas of research where the treatment cycling approach may be highly advantageous is T-cell research, specifically the study of peptide specificity of the T-cell receptors, TCRs. Genetic variability of the TCR is enormous; a single person may have up to 10⁸ different individual variants of the TCRs corresponding to individual T-cells or their clones. TCRs specifically (however, with some levels of cross-specificity) interact with the complexes of HLA molecules with peptides, usually short amino-acid sequences generated during the normal protein metabolism in the live cells. In the inflammatory context, TCR may recognize the corresponding non-self peptide presented as an HLA-peptide complex on the surface of the antigen-presenting cell. An outcome of this interaction can comprise T-cell activation, change of phenotype, proliferation, and cytokine secretion; the activated T cells help induce antigen-related antibody responses, participate in antigen-specific signaling, and kill the target cells exposing the non-self peptides on the surface. In many cases, it is enough to determine if any T-cells in the specimen can respond to any random parts of one or more antigens related to some pathogen or gene therapy vector. However, studying the precise peptide specificity of the TCRs and corresponding T-cells may be useful to optimize the response. For example, if simplified, a strong T-cell response would be beneficial in developing vaccines, cancer, and chronic infection immunotherapies. The “low-to-no” T-cell response would be important to demonstrate in developing cell and gene therapies, autoimmunity treatments, and allergy desensitization immunotherapies. Measuring T-cell responses may have high importance in both preclinical and clinical research, as well as in clinical practice as a diagnostic tool.

Taking into account the variability of both TCRs (10⁸) and peptides of the length relevant to HLA-peptide-TCR interaction, there may be only a vanishingly slight chance to activate even a single T-cell in a specimen using one single peptide that corresponds to the antigen of interest (protein). In such a case, not only is the chance of activation quite low, but there is also a massive waste of the specimen if the response is negative (and for patients, this specimen is usually blood drawn for research). As such, the treatment cycling approach described herein can be useful.

Another area where the treatment cycling approach may be advantageous is the research of rare primary cell types activation by the libraries of compounds with low chances of inducing the response of interest. Mixed primary human cell cultures are probably the most relevant ex vivo and in vitro research models able to capture the features of human-specific effects and diversity of human genetic background (as opposed to the use of experimental animals; or animal and human immortal cell lines). Major disadvantages of these models include the lack of standardization and questions regarding the sample's representativeness compared to the general population. Another problem is logistic access to the materials from many donors and the costs of such materials, in parallel with generally limited access to primary cells. The problem becomes even harder when the research has to be done in the rare cell types, which comprise only a small percentage of the total cell population in the tissue. An attempt to screen the extensive library of drug-like molecules (analytes, up to 1000 or more) to see if these molecules activate the rare cell type may be highly wasteful in the case one decides to treat the cell culture once and discard the specimen after the negative response is recorded (which may happen in over 99.99% of times).

If 100 cycles are allowable by the model, the treatment cycling approach described herein can help use the rare and primary cells with 100 times improvement in the efficiency of sample use, or 100 times improved sensitivity, or a combinational gain in sample use efficiency and sensitivity. If these rare cells have highly genetically diverse receptors of interest, a single specimen may comprise a mixture of cells from different patients (while immune responses between them are blocked, for example, immune cells and antibodies are depleted), and later the response features/cycles can be correlated with genetic signatures. Such a mixture of the primary cells from 10 patients may lead to an additional 10-fold increase in the bioassay efficacy, with Cycling leading to a 1000-fold increase in specimen use efficacy.

The present invention comprises the devices and methods to cycle the testing of the specimen reactivity against multiple analytes to increase the frequency of detecting low-probability positive responses. A device or a set of devices can have the following functionality: (a) allows addition of the treatment to the suspension of the cells of interest, (b) measures the cellular response readout as negative or positive if one or more cells activated, (c) removes the excess treatment from the cell suspension, and optionally replenishes it with the washing solution, (d) repeats the cycles of steps (a)-(c), optionally excludes the samples or wells with a positive response from the following cycles by programming individual control of the treatment exchange in this sample or well. As a result, if the chance of activating any single cell during one treatment cycle is low, the repeated treatment cycles substantially decrease the need in using large volumes of the biosample for the screening. This device and method advantageously can work with a limited sample supply, especially with the limited proportion of the responsive cells of interest within the sample, wherein their responses should be screened against multiple treatments with a low probability of positive response to any given treatment.

The invention encompasses (1) the treatment cycling approach allowing the repeated use of the same specimen, in comparison to the industry's traditional one treatment per one experimental specimen/well/vessel to assess the effects of this treatment, (2) use of the quick readouts to allow cycling in comparison to the industry's traditional readouts usually recorded in at least hours after the treatment, (3) use of various quick cell washes or treatment exchanges without centrifugation at high centrifugal forces, in comparison to the industry's traditional approach, (4) reliance on using the single-cell-generated responses and colonies, linked to the specific activating treatment conditions (numbers of cycles or coordinates or responding cell), for the sequencing of their features, in comparison to the industry's traditional alternatives of single-cell sequencing or bulk sequencing done for the unknown number of proliferated colonies without their individual treatment-response tracking.

This invention can utilize a limited specimen supply (limited by cost, logistics, ethical reasons, etc.), especially with the limited proportion of the responsive cells of interest within the sample, where their responses should be screened against multiple treatments with a low probability of positive response to any given treatment. An increase in the bioassay efficacy up to 100-1000 times compared to the traditional approaches can be expected. This increase in efficacy can (a) simplify the research logistics and decrease costs, as a result making previously impossible studies possible, (b) decrease the required volumes of specimens, making the research procedures less harmful or painful for the experimental animals or patients, (c) make the research procedures less wasteful improving the ecological outcomes of research, (d) stimulate new research areas such as the studies of T and B cells, studies of the rare cell types and the compounds (drugs, peptides) interacting with these cells and their receptors in the context of high biological and genetic diversity.

The invention is illustrated by the following non-limiting examples.

### EXAMPLES

### Example 1: Multiplex Testing for T-Cell Reactivation by Widespread Infections

The human frozen PBMCs from 3 donors pre-tested for high T-cell response (over 100 spots per 400,000 cells) to the influenza virus antigens by ELISpot are purchased (Cellular Technology Limited, OH, USA). PBMCs collected after 06/2022 are used to expect high T-cell response to the Covid antigens (post-vaccination and post-infection). Cells are thawed according to the general PBMC thawing protocol. Briefly, 9 mL of T cell medium (OpTmizer T Cell Expansion SFM, Gibco, with the addition of 1:100 GlutaMAX, Gibco) pre-warmed to +37° C. is prepared. The frozen cell vials are warmed in the water bath at +37° C. until ⅔ of the contents become liquid. Vial contents are quickly transferred to the tube with a warm medium and centrifuged at 300×g for 5 minutes at +20° C. The pelleted cells are resuspended in a T cell medium and used for assay immediately or plated at 1-2M/mL in 3 mL/well of a 6-well plate overnight in the CO2 incubator at +37° C.

Cells are separated, 200K in 200 ul of T cell medium for staining with green or red fluorescent dyes (Calbrite 520 AM and Calbrite 630 AM, ATT Bioquest, CA, USA), markers for the calcium flux, a quick event happening after T cell activation. These dyes diffuse and stay inside the cells; they are not fluorescent unless the T cell activates and the Ca++ ions concentration in the cytoplasm increases. The staining is done per manufacturer's protocols for 1 hour at +37° C. Then the cells are washed by centrifugation at 300×g for 5 minutes at +20° C. and resuspended in a T-cell medium. Unstained cells can be used as an additional negative control, demonstrating the potential autofluorescence before and after the T cell activation.

Cells in 100 ul of the T-cell medium are added to a transwell insert (Permeable cell culture inserts, 0.4 um pores, Celltreat, USA). The transwell insert is placed in the 10-20-ul drop of the T-cell medium (later referred to as the Drop) at the bottom of a 6-well plate. The plate is centrifuged at 250×g for 5 minutes at +20° C. to allow the concentration of cells at the bottom of the insert.

Imaging is done using the EVOS M7000 imager and green or red channels of fluorescence capable of registering the corresponding dye signals. The fluorescent settings are set up using the positive control sample that received the positive control treatment for 10 minutes at +37° C. Positive controls of two types are used: CytoStim, human (Miltenyi Biotek, USA), and Cell Stimulation Cocktail (eBioscience™, USA).

At different cycles, the Drop below the transwell insert is removed by pipetting. Then either (1) negative control, T cell medium, (2) the Flu antigenic peptide pool (PepTivator® Influenza A (H1N1) HA, Miltenyi Biotek, USA), or (3) the Covid antigenic peptide pool (PepTivator® SARS-COV-2 Prot_S1, Miltenyi Biotek, USA), or positive controls of two types are added at the same volume as the initial Drop under the transwell insert at the recommended or up to 5-fold higher concentration.

Gentle wash/treatment exchange in this example is an exchange of the liquid in the Drop, while the cells are staying in the T cell medium inside the transwell insert. The insert has pores of 0.4 um, and does not show signs of leaking when removed for exchange of the Drop. In this example, the process is manual and not automated. When the process is automated, a different method of gentle wash/treatment exchange can be used. The treatment cycles can include or omit the wash with the T cell medium between the activation cycles. The cycle can include the addition of the new analyte to the analyte of the preceding cycle and the mixing of them, instead of the removal of the previous analyte.

The stimulation peptides or positive controls interact with the cells via the transwell membrane. Cells are returned to the CO2 incubator or placed on the heated pad at +37° C. for 5-10 minutes after each stimulation. Alternatively, the plate heating system, an Onstage Incubator, can be used to keep the plate at +37° C. at most times. After that, the green or red signals are recorded. When multiple wells are used, the signal is obtained by the programmed scanning of the select areas of the plate using the corresponding green or red channel. At least several wells can be scanned in less than 1 minute.

The count of activated cells after they receive one or sequentially two antigenic peptide pools, or any type of positive controls, does not vary more than 5% based on the treatment cycle up to 10 or more cycles. For the consistent count, the cutoff values for the cell count should be selected, as well as consistent fluorescent imaging conditions.

In some examples, the treatment cycling can be stopped when a positive result is obtained (e.g., treatment with stopping). For example, after the response of the cells to one of the peptide pools is obtained, the data are recorded and the sample is not used for the further cycling. This approach can be used for comparing the consistency of cell numbers responding to the same peptide pool at different treatment cycles, for example, cycle #1 and cycle #10. In the case of one single peptide use, and if only a few cells respond to treatment, then the cells can be allowed to proliferate (optional) and can be isolated for T cell receptor (TCR) sequencing.

In additional examples the treatment cycling can continue when a positive result is obtained (treatment without stopping). In this example, even after some cells respond to one peptide (pool), the cycles are continued, to see if even more cells will respond to the other peptides (pools). In the “treatment without stopping” approach, the cells which de novo responded to the last cycle treatment in the simplest situation represent the number of fluorescent cells at the current readout cycle minus the number of fluorescent cells at the previous readout cycle (since the decrease of fluorescence after it was induced is a slow process and can take multiple hours). Since Ca++ signaling spike deteriorates with time, for multiple cycles more sophisticated data analysis workflow should be implemented, where every individual cell is traced and only newly originated activation events are counted for each cycle, while signals from the previously activated cells are ignored. This approach allows testing the T cell response of a patient sample against multiple antigens within the same sample. The quality control in the last cycle can be the addition of positive control.

Additionally, one or more of strategies to improve method specificity may be employed. A significant limitation when using Ca++ flux as a T-cell activation readout is the non-specific nature of this event in cellular signaling. This event is present when the apoptosis or phagocytosis is initiated, or different other cell types activate. The user of method can apply additional sensors for apoptosis and other non-specific events to improve the method specificity by excluding these events from counting. The user can sort or pre-label cell types to avoid counting non-specific events originating from irrelevant cell types. The user can select more specific methods to identify T-cell activation such as immune synapse formation. Even if all T-cell activation events captured during cycling are real, there will be T-cell populations non-secreting cytokines upon activation, and this should be accounted for.

### Example 2: Treatment Cycling With Downstream Elispot

Treatment cycling can be an assay run with the cells before the single-cytokine and multi-cytokine ELISpot of FLUOROSpot methods to detect the T cells secreting the cytokines after activation. Right after the cyclic treatments are done, data are recorded within 20 hours of the beginning of the treatments; the treated cell suspension can be transferred to the ELISPOT membrane coated with the antibody(ies). Cells in this assay are activated at different time points before placement on the membrane (preincubation), and the dynamics of cytokine secretion may vary based on this factor. One strategy to avoid the inconsistent timing of T-cell stimulation before adding them to the ELISpot membrane is using two cycling rounds. The first round will be done with one part of the sample, detecting the most immunogenic peptides (for example, 10 out of 100), and the second round will include fewer cycles before using the cells for ELISpot. Therefore the preincubation time difference will be minimized.

Another alternative is detecting the most immunogenic peptides and using their pool exclusively to activate the cells before the ELISpot. In this case, the preincubation time difference will be eliminated, and the epitope mapping data will still be obtained. Another alternative is simply running the Treatment Cycling and ELISpot in parallel. The overall number of activated T cells per 1 million PBMCs should be consistent or at least well correlated. One assay will provide epitope mapping data, while the other will provide information about the types of stimulation.

More complex alternatives allow the detection of the specific stimulatory peptide and type of T-cell activation simultaneously. However, these would be too different from the ELISpot and not discussed in this section.

The 20 hours of preincubation may be considered too long, and represent a non-standard approach. However, this is known that for detecting some cytokines, such as IL-2, cells should be preincubated after stimulation for optimal ELISpot results. Moreover, in the cytokine secretion assays, 48 and 72 hours after the T-cell stimulation are the preferred time points compared to 24 hours, indicating high cytokine secretion activity after 24 hours. Despite differences in the preincubation period for the cells activated at different cycles, we expect Cycling-ELISpot with 100 different peptides vs. regular ELISpot with a peptide pool of 100 peptides to correlate well (with proper exclusion of false-positive activation events, or more precise inclusion of real T cell activation events). Moreover, the results of Cycling-ELISpot will be supported by the expected spot quantity after cycling (sum of all cells stimulated at all cycles). And the former will give higher data resolution and provide the T cell epitope mapping data, compared to the regular ELISpot giving only the overall results of the T cell stimulation by the peptide mixture. Both approaches would require at least one well of negative and one well of positive controls.

### Example 3: Treatment Cycling With Downstream ICS-FACS

Treatment cycling can be an assay run with the cells before the single-cytokine and multi-cytokine ICS-FACS to detect the T cells secreting the cytokines after activation. ICS-FACS is a flow cytometry-based method to detect intracellular cytokines, usually for detecting antigen-specific T cells. It requires T-cell stimulation and 2 hours of preincubation in some implementations, after which the Golgi apparatus blocking reagent is added to the cells. After 4 hours with the blocking reagent, the cells are stained for viability, surface markers, and intracellular cytokines using fluorescent dyes and fluorescently labeled antibodies. In this assay, the standard 2 hours of pre-incubation can be substituted with 20 hours of cycling without the Golgi apparatus blocking agent, plus an additional 2 hours of pre-incubation without cycling to allow the cells stimulated at the latest cycles to upregulate the cytokine expression (as in standard approach). The results of the cytokine secretion will be partially affected by the time difference of T-cell stimulation (actual pre-incubation times will be different for the cells stimulated at different cycles). Strategies to overcome this limitation are discussed in the Cycling-ELISpot methods combination section. Similar strategy can be employed to detect the cells with upregulated activation-induced markers (AIM) with the longer post-cycling incubation timing before the cell staining and analysis (FACS-AIM).

### Example 4: Treatment Cycling With Downstream Cytokine Secretion Assay

Treatment cycling can be an assay run with the cells before detecting the cytokines secreted by the T cells after activation. This combination is probably the simplest. After all, cycles are done, cells are placed inside the CO2 incubator for another 24-48 hours. Many multiplex cytokine detection methods, such as LEGENDplex, Luminex, and MSD (Meso Scale Discovery), can detect secreted cytokines. If the cycling is run so that only one cell is activated per well, and the cycling stops-in this case, the longer incubation time should be recommended, and the preference should be given to more sensitive methods, such as MSD.

### Example 5: Treatment Cycling With Downstream Cell Proliferation and TCR Sequencing

Treatment cycling can be done before letting the T cells proliferate after stimulation. Proliferation is one of the oldest T cell activation detection methods. This usually requires 5-7 days or longer. Current instrumentation allows automatic colony counting and picking if necessary. Bulk RNA sequencing will allow finding the TCR sequence if only one colony is proliferating per well (especially, if the used well is very small). Even if several colonies are generated per well from the cells activated at the known cycles, in silico prediction methods may allow finding what activating peptides correspond to what TCRs. The pre-staining of the cells with oligonucleotide-barcoded antibodies and with MHC/HLA multimers pre-loaded with the antigenic peptides (optionally barcoded) may allow to pre-sort the cells, control the cells'phenotype, and run single-cell sequencing.

### Example 6: Test of Repeated T-Cell Activation Protocol With Green Dye Staining

The cell source was a vial of human frozen PBMCs from 1 donor pre-tested for high T-cell response (>100 spots per 400,000 cells) to the influenza virus antigens by ELISpot (Cellular Technology Limited, OH, USA). PBMCs collected after June 2022 are used to expect high T-cell response to the Covid antigens (post-vaccination or post-infection). Cells were thawed according to the general PBMC thawing protocol. Briefly, 9 mL of T cell medium (OpTmizer T Cell Expansion SFM, Gibco, with the addition of 1:100 GlutaMAX, Gibco) pre-warmed to +37° C. is prepared. The frozen cell vials were warmed in the water bath at +37° C. until ⅔ of the contents became liquid. Vial contents were quickly transferred to the tube with a warm medium and centrifuged at 300×g for 5 minutes at +20° C. The cells are resuspended in a T cell medium and plated at the concentration 1-2M/mL, 3 mL/well of a 6-well plate overnight in the CO2 incubator at +37° C. Cells were separated, 200K in 200 ul of T cell medium for staining with green fluorescent dye Calbrite 520 AM (ATT Bioquest, CA, USA), for 1 hour in the CO2 incubator at +37° C. Then the cells were washed by centrifugation at 300×g for 5 minutes at +20° C. and resuspended in a T-cell medium.

Cells, 100K in 100 ul of the T-cell medium, were added to a transwell insert (Permeable cell culture inserts, 0.4 um pores, Celltreat, USA). The bottom of the transwell insert was placed in the 20-ul drop of the T-cell medium (later referred to as the Drop) at the bottom of a well of a 6-well plate. Imaging was done using the EVOS M7000 imager at a green fluorescence channel. At different cycles, the Drop below the transwell insert was removed by pipetting. Then either (1) negative control, T cell medium, (2) the Flu antigenic peptide pool (PepTivator® Influenza A (H1N1) HA, Miltenyi Biotek, USA), or (3) the Covid antigenic peptide pool (PepTivator® SARS-COV-2 Prot_S1, Miltenyi Biotek, USA), or positive controls of two types are added at the same volume as the initial Drop under the transwell insert at the 5-fold higher concentration. Positive controls of two types are used: CytoStim, human (Miltenyi Biotek, USA), and Cell Stimulation Cocktail (eBioscience™, USA). For treatment cycling, the liquid in the Drop was exchanged for the next treatment type while the cells stayed in the T cell medium inside the transwell insert (manually removed to exchange the drop). The insert has pores of 0.4 um, and did not show signs of leaking when removed for exchange of the Drop. There was no wash between the treatment cycles in this experiment. The stimulation peptides or positive controls interact with the cells via the transwell membrane. Cells were returned to the CO2 incubator at +37° C. for 8 minutes after each stimulation. After that, they were imaged, and green signals were observed. Five treatment cycles were performed, and the step-by-step increase in fluorescent spot (cell) numbers was recorded. T cell activation function immediately post-staining was confirmed, as well as the green dye suitability in the T cell activation experiments. An analogous experiment was performed with the red dye Calbrite 630 AM (Example 7).

### Example 7: Test of Medium Exchange Protocol and Red-Dye Staining

The cell source was a vial of human frozen PBMCs from 1 donor pre-tested for high T-cell response (>100 spots per 400,000 cells) to the influenza virus antigens by ELISpot (Cellular Technology Limited, OH, USA). PBMCs collected after June 2022 are used to expect high T-cell response to the Covid antigens (post-vaccination or post-infection). Cells were thawed according to the general PBMC thawing protocol. Briefly, 9 mL of T cell medium (OpTmizer T Cell Expansion SFM, Gibco, with the addition of 1:100 GlutaMAX, Gibco) pre-warmed to +37° C. is prepared. The frozen cell vials were warmed in the water bath at +37° C. until ⅔ of the contents became liquid. Vial contents were quickly transferred to the tube with a warm medium and centrifuged at 300×g for 5 minutes at +20° C. The cells were resuspended in a T cell medium and plated at the concentration 1-2M/mL, 3 mL/well of a 6-well plate overnight in the CO2 incubator at +37° C.

Cells were separated, 200K in 200 ul of T cell medium for staining with red fluorescent dye Calbrite 630 AM (ATT Bioquest, CA, USA), for 1 hour in the CO2 incubator at +37° C.

Then the cells are washed by centrifugation at 300×g for 5 minutes at +20° C. and resuspended in a T-cell medium. Cells were placed in the 2-ml capped tube with a slightly unscrewed cap for ventilation, and incubated for 24 hours in the CO2 incubator at +37° C.

Cells, 100K in 100 ul of the T-cell medium, were added to a transwell insert (Permeable cell culture inserts, 0.4 um pores, Celltreat, USA). The bottom of the transwell insert was soaked in the 200-ul of the T-cell medium at the bottom of a well of a 24-well plate. Imaging was done using the EVOS M7000 imager at a red fluorescence channel.

At different cycles, the medium below the transwell insert was removed by pipetting. Then either (1) negative control, T cell medium, (2) the Flu antigenic peptide pool (PepTivator® Influenza A (H1N1) HA, Miltenyi Biotek, USA), or (3) the Covid antigenic peptide pool (PepTivator® SARS-COV-2 Prot_S1, Miltenyi Biotek, USA), or positive controls of two types were added at the same volume under the transwell insert at the recommended concentration.

Positive controls of two types are used: CytoStim, human (Miltenyi Biotek, USA), and Cell Stimulation Cocktail (eBioscience™, USA). For treatment cycling, the liquid under the insert was exchanged for the next treatment type while the cells stayed in the T cell medium inside the transwell insert (staying inserted in the plate). There was no wash between the treatment cycles in this experiment. The stimulation peptides or positive controls interact with the cells via the transwell membrane. Cells are returned to the CO2 incubator at +37° C. for 8 minutes after each stimulation. After that, they were imaged, and red signals were observed. Five treatment cycles were performed, and the step-by-step increase in fluorescent spot (cell) numbers was recorded. T cell activation function 24 hours post-staining was confirmed, as well as the red dye suitability in the T cell activation experiments that take over 24 hours. The representative pictures showing the increase of activated T cells responding to Flu and Covid peptide treatments between two treatment cycles are shown in FIG. 17. The activated cells are outlined and counted using the free software package QuPath. These proof-of-concept data are not cleaned of the potential false-positive events, and methods for data cleaning are discussed elsewhere.

### Example 8: Test of Fully Automated System of Analyte Fluid Exchange and Imaging

The automated “new treatment” transfer between the analyte fluid library and transfer plate was realized as the liquid handling by the OT-2 Robot (Opentrons, USA) programmed via PC. Automated scheduled analyte fluid transfer in 12-channel format between the Transfer plate and Test plate containing the biosample was realized by multiplex tubing and multiple diaphragm pumps switched on and off by the relay and microcontroller programmed via PC. Automated scheduled analyte fluid removal from the Test plate was realized by multiplex tubing connected to the vacuum pump switched on and off by the relay and microcontroller programmed via PC. Automated scanning of the pre-selected regions of the pre-selected wells of the multiwell plate was realized by using the microscopes with automated stage (BZ-X800, Keyence, Japan) or automated optical system moving below the plate (Celloger Mini Plus, Curiosis, Korea), programmed via PC. The implemented automated equipment set is depicted by the scheme shown in FIG. 11.

### Example 9: Test of Aimspot Method for Staining Aim Markers or Intracellular Cytokines Via the Membrane of Transwell Insert

AIMspot is a method that allows combination of treatment cycling with automated or semi-automated immunofluorescent staining or other imaging techniques presenting the final outcomes of the activation cycling. Spatial data overlap allows removal of false positive activation events, and confirms true positives. For the true positive events, cycling data shows how many cells were activated (yes/no) by any specific analyte fluid, and the downstream part of AIMSpot provides the data on cellular phenotypes and activation outcomes.

In this example, the cell source was a vial of human frozen PBMCs from 1 donor. Cells were thawed according to the general PBMC thawing protocol. Briefly, 9 mL of T cell medium (OpTmizer T Cell Expansion SFM, Gibco, with the addition of GlutaMAX and Glutamine, Gibco) pre-warmed to +37° C. is prepared. The frozen cell vials were warmed in the water bath at +37° C. until ⅔ of the contents became liquid. Vial contents were quickly transferred to the tube with a warm medium and centrifuged at 300×g for 5 minutes at +20° C. The cells were resuspended in a T cell medium and plated inside the transwell inserts, 100-150K per insert in the volume of 50-100 ul, with 300 ul of cell culture medium below the insert. In the test wells, the medium was replaced with the medium containing the pool of Flu peptides (as described in previous examples). In the positive control wells, medium was replaced with the medium containing the Cell Stimulation Cocktail (eBioscience™, USA).

For study of the phenotypic and AIM markers, the cells were pre-loaded with Ca++ sensor and imaged post-activation (as discussed in earlier examples; only 1 cycle of treatment and Ca++ readout in this example), and the plate was left for 24 hours post-stimulation in the CO2 incubator at +37° C. (AIM markers usually appear on the surface of activated cells 24-48 hours after antigen-specific stimulation). Residual fluorescence of Ca++ sensor in red channel was eliminated by 30 minutes of photobleaching under the LED lights; exchange of the fluid below cells to Ca-free solutions also helped completely remove Ca++ sensor signals.

The staining with fluorescent antibodies is performed without centrifugation, by exchanging the medium below the transwell insert. Blocking before the antibody staining was performed for 20 minutes at room temperatures with the blocking buffer A containing 2% FBS and Fc Receptor Blocking Solution (Human TruStain FcX, Biolegend, USA) in Ca-free PBS, 300 ul per well. Antibody staining was performed by using 1×-3× of the manufacturer's recommended antibody concentrations in the blocking buffer. In this specific example phenotypic marker was CD4, and AIM markers included CD69 and 4-1BB. Antibodies used for staining included anti-human CD4 Alexa Fluor 488-labeled antibody, anti-human CD69 PE-labeled antibody and anti-human 4-1BB PE-labeled antibody. Staining was performed for 40 minutes at room temperature. Samples were washed by exchanging the fluid below transwell with PBS, 3 times for 20 minutes at room temperature. Imaging was performed using the microscope BZ-X800 (Keyence, Japan). Green CD4+ and red (CD69/4-1BB)+cells can be discriminated (FIG. 18, top two images).

For study of intracellular protein expression (IFN-γ), only the downstream part of the AIMSpot protocol was tested in this example (no Ca++ signaling imaging but methods of performing such imaging are described above). The same cells without pre-loading of Ca++ sensor were plated in the same transwell inserts and treated only with positive control treatment or left untreated as a negative control. The plate was left for 2 -12 hours at +37C. The medium in the bottom chamber of the transwell was exchanged in a semi-automated format for the medium containing Brefeldin A and left for additional 4 hours of incubation in the CO2 incubator at +37° C. Then the cellular fixation and permeabilization was performed using the eBioscience™ Foxp3 /Transcription Factor Staining Buffer Set according to the manufacturer's protocol with major modifications: (1) the centrifugation and cellular resuspension steps are substituted with the semi-automated fluid exchange below the transwell membrane with additional incubation time to allow diffusion, (2) cell resuspension in permeabilization buffer is substituted with 20 minutes of incubation at room temperature with this buffer below the transwell insert (Perm-wash step), (3) blocking is performed with blocking buffer B containing 2% FBS and Fc Receptor Blocking Solution (Human TruStain FcX, Biolegend, USA) in permeabilization buffer, added below the transwell insert for 20 minutes of incubation at room temperature, (4) staining is performed after blocking by adding the primary mouse IgG antibody against human IFN-γ below the transwell insert for 2 hours at room temperature, 2 additional Perm-wash steps, additional blocking step, and by adding the secondary anti-mouse IgG antibody labeled with Alexa Fluor 647 below the transwell insert for 1 hour at room temperature. The cells were imaged after 3 additional Perm-wash steps, and the last wash step that substituted the fluid below the transwell insert for PBS. Cells were imaged using Nikon Ti2 inverted fluorescent microscope; positive control shows plenty of intracellular fluorescent INF-γ spots (FIG. 18, bottom four images; transmitted light images demonstrate the cell density on the top of transwell membrane).

All references, articles, patent applications, patent publications and patents are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of detecting T-cell activation, the method comprising:
i. a first cycle comprising:
a. contacting a sample comprising T-cells with an analyte fluid, wherein the sample comprising the T-cells is on a solid support and wherein the analyte fluid comprises a candidate agent in a liquid medium;
b. detecting the T-cell activation by detection of an optical signal in the sample on the solid support; and
c. removing the analyte fluid from the solid support thereby removing the candidate agent while retaining the sample comprising the T-cells on the solid support;
ii. a subsequent cycle comprising:
a. contacting the retained sample comprising the T-cells of the preceding cycle with a new analyte fluid, wherein the new analyte fluid comprises a new candidate agent in a liquid medium, wherein the new candidate agent is different from the candidate agent of the preceding cycle;
b. detecting the T-cell activation by detection of an optical signal in the retained sample on the solid support; and
c. removing the analyte fluid from the solid support thereby removing the candidate agent while retaining the sample comprising the T-cells on the solid support;
repeating the subsequent cycle at least N times, wherein N is an integer greater than or equal to 0, wherein each new analyte fluid of each subsequent cycle comprises a different candidate agent from that of the preceding cycle;
wherein the removal of the analyte fluid is a method whereby the cell viability and/or the cell quantity of the retained sample would be decreased by no more than about 5% after 10 total cycles, and that does not comprise centrifugation.

2. The method of claim 1, wherein the sample comprising T-cells is multiple samples comprising T-cells and
wherein the solid support is a multi-well plate and wherein the multiple samples comprising T-cells are in multiple wells of the plate; or
wherein the solid support is a transwell insert, wherein multiple transwell inserts are placed in wells of a multi-well plate, wherein the multiple samples comprising T-cells are on the multiple transwell inserts, and wherein the analyte fluid is added to the wells of the multi-well plate.

3. The method of claim 2, wherein the samples in at least two of the wells or at least two of the inserts are contacted with different candidate agents in the first cycle and/or in a subsequent cycle.

4. The method of claim 3, wherein the samples in each of the wells or in each of inserts is contacted with a different candidate agent in the first cycle and/or in a subsequent cycle.

5. The method of claim 1, wherein N is 5 or more.

6.-9. (canceled)

10. The method of claim 3, wherein at least one candidate agent is a positive control.

11. The method of claim 3, wherein at least one candidate agent is a negative control.

12. The method of claim 1, wherein the candidate agent is a library of candidate agents.

13. The method of claim 1, wherein the candidate agent is selected from the group consisting of a small molecule or a biologic.

14. The method of claim 13, wherein the biologic is an antibody or a peptide.

15.-16. (canceled)

17. The method of claim 1, wherein the candidate agent is a bacterial antigen, a viral antigen, or a cancer antigen.

18. (canceled)

19. The method of claim 4, wherein the candidate agent is a peptide that is 8-24 amino acids in length, a peptide that is 8-15 amino acids in length, or a peptide that is 8-12 amino acids in length.

20. The method of claim 1, wherein the optical signal indicates calcium flux, cytokine release, or the formation of a complex comprising a T-cell and an antigen-presenting cell (APC).

21. The method of claim 20, wherein the optical signal indicates calcium flux and the signal is the fluorescence of a calcium binding moiety that comprises a fluorescent label.

22. The method of claim 20, wherein the optical signal indicates formation of a complex between a T-cell and an APC and the signal is a change in cell shape.

23. The method of claim 1, wherein the optical signal is detected less than 6 hours after the sample is contacted with the analyte fluid.

24.-26. (canceled)

27. The method of claim 1, wherein the method or a portion thereof is automated.

28. The method of claim 1, wherein the method is high-throughput.

29. The method of claim 1, wherein the sample comprising T cells comprises PBMCs.

30. (canceled)

31. The method of claim 1, wherein the sample comprising the T cells further comprises antigen presenting cells.

32.-33. (canceled)

34. The method of claim 1, wherein the T cells comprise CD8+ T-cells.

35. The method of claim 1, wherein the T cells comprise CD4+ T-cells.

36. The method of claim 1, wherein a sample positive for T-cell activation in a cycle is not subjected to a subsequent cycle.

37. The method of claim 1, wherein a sample positive for T-cell activation in a cycle is subjected to a subsequent cycle.

38. The method of claim 1, wherein removal of the analyte fluid is by laminar cell wash.

39. The method of claim 2, wherein the solid support is a transwell insert, wherein multiple transwell inserts are added to wells of a multi-well plate, wherein the multiple samples comprising T-cells are on the multiple transwell inserts, and wherein the analyte fluid is added to the wells of the multi-well plate.

40.-41. (canceled)

42. The method of claim 1, wherein a sample positive for T-cell activation in any cycle is further analyzed by downstream assay.

43.-48. (canceled)

49. A method of identifying a cellular response to a candidate agent, the method comprising:
i. a first cycle comprising:
a. contacting a sample comprising cells with an analyte fluid, wherein the sample comprising the cells is on a solid support and wherein the analyte fluid comprises a candidate agent in a liquid medium;
b. detecting the response by detection of an optical signal in the sample on the solid support; and
c. removing the analyte fluid from the solid support thereby removing the candidate agent while retaining the sample comprising the cells on the solid support;
ii. a subsequent cycle comprising:
a. contacting the retained sample comprising the cells of the preceding cycle with a new analyte fluid, wherein the new analyte fluid comprises a new candidate agent in a liquid medium, wherein the new candidate agent is different from the candidate agent of the preceding cycle;
b. detecting the response by detection of an optical signal in the retained sample on the solid support; and
c. removing the analyte fluid from the solid support thereby removing the candidate agent while retaining the sample comprising the cells on the solid support;
repeating the subsequent cycle at least N times, wherein N is an integer greater than or equal to 0, wherein each new analyte fluid of each subsequent cycle comprises a different candidate agent from that of the preceding cycle;
wherein the removal of the analyte fluid is a method whereby the cell viability and/or the cell quantity of the retained sample is decreased by no more than about 5% after 10 total cycles and that does not comprise centrifugation.

50. The method of claim 49, wherein the cellular response is selected from the group consisting of activation or cell death.

51. A non-transitory computer-readable medium storing the computer-readable instructions which, when executed by one or more processors, manage the automated cycling operations of the method of claim 1.

52. (canceled)

53. An automated system or device for performing the method of claim 1, wherein the solid support is a well of a multi-well plate or a transwell insert placed in a well of a multi-well plate, and wherein the system comprises:
a. a dispensing unit configured for dispensing the analyte fluid into each well of the multi-well plate;
b. an optical imaging unit configured for detecting the activated T-cell by detection of an optical signal; and
c. a washing unit configured for removing the analyte fluid from each well thereby removing the candidate agent and retaining the sample comprising the T-cells in the well.

54. (canceled)

55. An automated system for detecting T-cell activation, wherein samples comprising T-cells are in the wells of a multi-well plate or on transwell inserts placed in the wells of a multi-well plate, the system comprising:
a. a dispensing unit configured for dispensing an analyte fluid into each well of the multi-well plate, wherein the analyte fluid comprising a candidate agent and a liquid medium;
b. an optical imaging unit configured for detecting an activated T-cell by detection of an optical signal; and
c. a washing unit configured for removing analyte fluid from each well thereby removing the candidate agent and retaining the sample comprising the T-cells in the well.

56-75. (canceled)
